SEQUENCE LISTING (2 sequences)

SEQ ID NO: 1 - protein, 433 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGASNRGPRNQFITHD

SEQ ID NO: 2 - protein, 448 aa, Homo sapiens
MLLPWATSAPGLAWGPLVLGLFGLLAAAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2013/0216557 A1
(43) Pub. Date: Aug. 22, 2013

(54) LTBR BLOCKADE: METHODS FOR OPTIMIZING THERAPEUTIC RESPONSIVENESS OF PATIENTS

(71) Applicants: Jadwiga Bienkowska, Cambridge, MA (US); Jeffrey L. Browning, Brookline, MA (US)

(72) Inventors: Jadwiga Bienkowska, Cambridge, MA (US); Jeffrey L. Browning, Brookline, MA (US)

(73) Assignee: Biogen Idec MA Inc., Cambridge, MA (US)

(21) Appl. No.: 13/816,691

(22) PCT Filed: Aug. 15, 2011
(86) PCT No.: PCT/US11/47787

§ 371 Date: Apr. 25, 2013

Related U.S. Application Data
(60) Provisional application No. 61/373,813, filed on Aug. 14, 2010.

(51) Int. Cl. C12Q 1/68

(52) U.S. Cl. CPC C12Q 1/6883
USPC 424/172.1; 436/501; 506/9; 435/7.1; 514/1.1

(57) ABSTRACT

The invention provides compositions and methods for predicting therapeutic responsiveness of a subject having an autoimmune disorder to an agent that inhibits signaling via LTβR based on the level of expression of IFN or a marker thereof in the subject. The invention also provides methods of treating selected subjects with agents that inhibit or reduce signaling vial LTβR.

### BACKGROUND OF THE INVENTION

Regulation of the Lymphotoxin (LT) signaling system plays an important role in the function of the immune system and is also involved in many aspects of immune diseases (Browning J L (2008) Immunol Rev 223:202-220). Interaction between the LTα/β hetero-trimer and the LTβ receptor (LTβR) are required for lymph node development and ectopic organization of lymphoid tissue. Furthermore, interactions between an additional cytokine called LIGHT and its receptors: LTβR, DcR3 and HVEM, are involved in T cell survival, pro-inflammatory events and potentially communication with dendritic cells. LT cc/13 belongs to the Tumor Necrosis Factor (TNF)-like family of cytokines and although there is some overlap between the lymphotoxin and TNF systems, they utilize distinct signaling systems (Ware C F (2005) Annu Rev Immunol 23:787-819).

Several agents known in the art have been used to inhibit signaling via LTβR. One of these, an LTβR fusion protein, has been used in trials to treat subjects having rheumatoid arthritis who were Methotrexate or TNF inadequate responders. However, to date there is no way of identifying subjects that have an increased likelihood of responding to such therapy. The discovery of the biomarkers to identify patients for whom LTβR blockade will be effective would allow targeted drug administration, limit exposure of patients to ineffective drugs and allow informed selection of alternative therapies.

### SUMMARY OF THE INVENTION

The present invention is based, at least in part, on the surprising finding that the presence of elevated interferon (IFN) levels in a subject having an autoimmune disorder is predictive of improved responsiveness to treatment with an agent that blocks LTβR signaling.

The IFN signaling system is central to innate immunity and has been shown to play an important role in many auto-immune diseases. Increased levels of IFN can be measured directly or can be detected based on downstream effects. For example, reporter cell lines that give a specific quantifiable signal upon exposure to low levels of IFN (e.g. Wekerele et al 2011, Arthritis & Rheumatism 63:1044) can be used to detect IFN levels. Typically, serum or plasma from the patient is incubated with the reporter lines to determine whether there is IFN present.

In addition, various markers of IFN are also known in the art and can be detected in lieu of directly detecting IFN levels. For example, IFN-inducible gene expression signature patterns have been identified. Up-regulation of IFN responsive genes is a molecular signature present in many autoimmune diseases. For example, IFN-inducible genes are up-regulated in about 50% of patients with Systemic Lupus Erythomatosus (SLE) and with varying frequency in many autoimmune diseases. In some studies, the presence of an IFN signature has been linked to the severity of the disease. IFNs come in three basic types, I, II and III. As a type I response can lead to the production of type II IFN, a precise set of gene induction patterns cannot be readily assigned to a type I IFN response and, most likely, depending on the setting, all three types can contribute to the IFN signature. Furthermore, increased levels of chemokines and autoantibodies can be used as markers of increased levels of IFN.

Although markers of increased IFN levels have been suggested for use as pharmacodynamic biomarkers to aid in dose selection for other agents (e.g., for dose selection anti-IFNα mAb for SLE patients; Yao et al. 2010. Arthritis Research Therapy 12:56), it was not known that elevated expression of such markers, e.g., certain IFN-inducible genes correlates with the increased response to LTβR blockade in patients having an autoimmune disorder.

As shown by the data set forth herein, the presence of increased levels of IFN, e.g., as demonstrated by an elevated interferon-inducible gene expression signature in a subject having an autoimmune disorder, is predictive of responsiveness to treatment with an agent that reduces LTβR signaling. Prior to the instant invention there was no teaching or suggestion in the art that subjects having elevated levels of IFN or downstream markers thereof, (e.g., increased levels of autoantibodies, increased levels of interferon-inducible genes (e.g., an IFN expression signature), or increased levels of chemokines) would respond more favorably than those subjects not having such a gene expression profile to treatment with an agent that inhibits LTβR signaling.

In one aspect, the invention pertains to a method for predicting the responsiveness of a subject having an autoimmune disorder to a treatment with an agent that inhibits LTβR-mediated signaling, the method comprising, contacting a biological sample from the subject with a reagent allowing detection of increased levels of IFN or a marker thereof; detecting the level of IFN or a marker thereof, wherein the presence of an increased level of IFN or a marker of the expression thereof as compared to an appropriate control indicates that the patient will likely respond to therapy with an agent that inhibits LTβR-mediated signaling.

In one embodiment, the biological sample from the subject is contacted with a reagent to obtain a detectable composition allowing detection of the level of IFN or a marker thereof.

In one embodiment, increased levels of IFN are detected.

In one embodiment, increased levels of one or more genes induced by IFN are detected.

In one embodiment, increased levels of one or more autoantibodies are detected.

In one embodiment, increased levels of one or more cytokines are detected.

In one embodiment, the method further comprises treating the subject with an agent that inhibits LTβR-mediated signaling.

In one embodiment, the biological sample is a plasma sample.

In one embodiment, the biological sample is a blood sample.

In one embodiment, the biological sample comprises cells.

In one embodiment, the biological sample is manipulated prior to the step of contacting.

In one embodiment, the biological sample comprises extracted nucleic acid molecules.

In one embodiment, the reagent comprises a nucleic acid molecule which hybridizes to a nucleic acid molecule derived from the transcript of at least one gene induced by IFN.

In one embodiment, the at least one gene is selected from the group consisting of: OAS3, HERC5, OAS1, TIMM10, RSDA2, IFI44L, IFI44, IFI6, IFIT3, ISG15, MXI, DOX58, UBE2L6, BATF2, and LIPA.

In one embodiment, the level of expression is determined by measuring transcription of a plurality of genes induced by IFN.

In one embodiment, the level of transcription of at least 5 genes induced by IFN is detected.

In one embodiment, the level of transcription of at least 10 genes induced by IFN is detected.

In one embodiment, the level of transcription of the OAS3, HERC5, OAS1, TIMM10, RSDA2, IFI44L, IFI44, IFI6, IFIT3, ISG15, MXI, DOX58, UBE2L6, BATF2, and LIPA genes are detected.

In one embodiment, the level of expression of at least one chemokine in the subject is measured.

In one embodiment, the method further comprises measuring the level of expression of at least one chemokine in the subject.

In one embodiment, the at least one chemokine is selected from the group consisting of CXCL10, CCL19, and CCL2.

In one embodiment, the reagent comprises a reporter cell.

In one embodiment, the reagent is capable of detecting the presence of an autoantibody.

In one embodiment, the autoimmune disorder is selected from the group consisting of: rheumatoid arthritis, Sjogren's syndrome, scleroderma, lupus, polymyositis/dermatomyositis, cryoglobulinemia, anti-phospholipid antibody syndrome, and psoriatic arthritis), autoimmune gastrointestinal and liver disorders, autoimmune gastritis and pernicious anemia, autoimmune hepatitis, primary biliary cirrhosis, primary sclerosing cholangitis, celiac disease, vasculitis, autoimmune neurological disorders, renal disorders, autoimmune dermatologic disorders, hematologic disorders, atherosclerosis, uveitis, autoimmune hearing diseases, Behcet's disease, Raynaud's syndrome, dermatomtositis, organ transplant, autoimmune endocrine disorders, IBD, and Type I diabetes.

In one embodiment, the autoimmune disorder is selected from the group consisting of: RA, Sjogren's syndrome, lupus, inflammatory myositis, psoriasis, multiple sclerosis, and rheumatoid arthritis.

In another aspect, the invention pertains to a method for treating a subject having an autoimmune disorder with an agent that blocks LTβR-mediated signaling, the method comprising, contacting a biological sample from the subject with a reagent allowing detection of increased levels of IFN or a marker thereof; detecting the level of IFN or a marker thereof, wherein the presence of an increased level of IFN or a marker thereof as compared to an appropriate control indicates that the patient will likely respond to therapy with an agent that inhibits LTβR-mediated signaling; selecting a patient having increased levels of IFN or a marker thereof for treatment with an agent that inhibits LTβR-mediated signaling.

In one embodiment, the method further comprises administering an agent that inhibits LTβR-mediated signaling to the subject.

In one embodiment, the agent that blocks LTβR-mediated signaling is selected from the group consisting of: a soluble form of an LTβ receptor, and antibody which binds to the LTβ receptor, and an antibody which binds to cell surface LTβ, an antibody that binds to LTa and inhibits the binding of LTβR to cell surface LTα.β, a form of the soluble decoy receptor DcR3 that reduces the binding of LIGHT to LTβR.

In another aspect, the invention pertains to a method for evaluating the response of a subject having an autoimmune disorder to treatment with an agent that blocks LTβR -mediated signaling, the method comprising, contacting a biological sample from the subject with a reagent allowing detection of increased levels of IFN or a marker thereof; detecting the level of IFN or a marker thereof, wherein the presence of an increased level of IFN or a marker of the expression thereof as compared to an appropriate control indicates that the patient will likely respond to therapy with an agent that blocks LTβR-mediated signaling; administering an agent that blocks LTβR-mediated signaling to the subject; contacting a second biological sample from the subject taken after step c) with a reagent allowing detection of increased levels of IFN or a marker thereof; wherein the presence of a decreased level of the detectable composition in the sample as compared to the level obtained in step a) indicates that the patient will likely respond to therapy with an agent that blocks LTβR-mediated signaling.

In yet another aspect, the invention pertains to a method of selecting a treatment regimen for a subject having an autoimmune disorder to a treatment with an agent that inhibits LTβR-mediated signaling, the method comprising, contacting a biological sample from the subject with a reagent allowing detection of increased levels of IFN or a marker thereof; detecting the level of IFN or a marker thereof, wherein the presence of an increased level of IFN or a marker of the expression thereof as compared to an appropriate control indicates that the patient will likely respond to therapy with an agent that inhibits LTβR-mediated signaling; selecting a treatment regimen for the subject employing an agent that blocks LTβR-mediated signaling.

In another aspect, the invention pertains to use of an agent that blocks LTβR-mediated signaling for treatment of an autoimmune disorder in a subject, wherein the subject exhibits increased levels of IFN or a marker thereof.

In still another aspect, the invention pertains to use of an agent that blocks LTβR-mediated signaling in the manufacture of a medicament for treatment of an autoimmune disorder in a subject, wherein the subject exhibits increased levels of IFN or a marker thereof.

In yet another aspect, the invention pertains to a kit for predicting therapeutic responsiveness of a subject afflicted with an autoimmune disorder to an agent that blocks LTβR-mediated signaling, the kit comprising a means for determining the level the level of IFN or a marker thereof in a biological sample from the subject.

In one embodiment, the kit comprises a means of detecting at least one gene regulated by IFN in a biological sample obtained from said subject and instructions for using the kit to predict therapeutic responsiveness of the subject having an autoimmune disorder to an agent that blocks LTβR-mediated signaling.

In one embodiment, the kit comprises a means for determining the level of a plurality of genes regulated by IFN in the sample.

### BRIEF DESCRIPTION OF THE DRAWINGS

FIG. 1 shows the lymphocyte counts present in subjects in from two groups of rheumatoid arthritis (RA) patients, RA202 and RA203. In both groups of subjects, there is a correlation between IFN positive status (x-axis) and decreased lymphocyte counts (y axis).

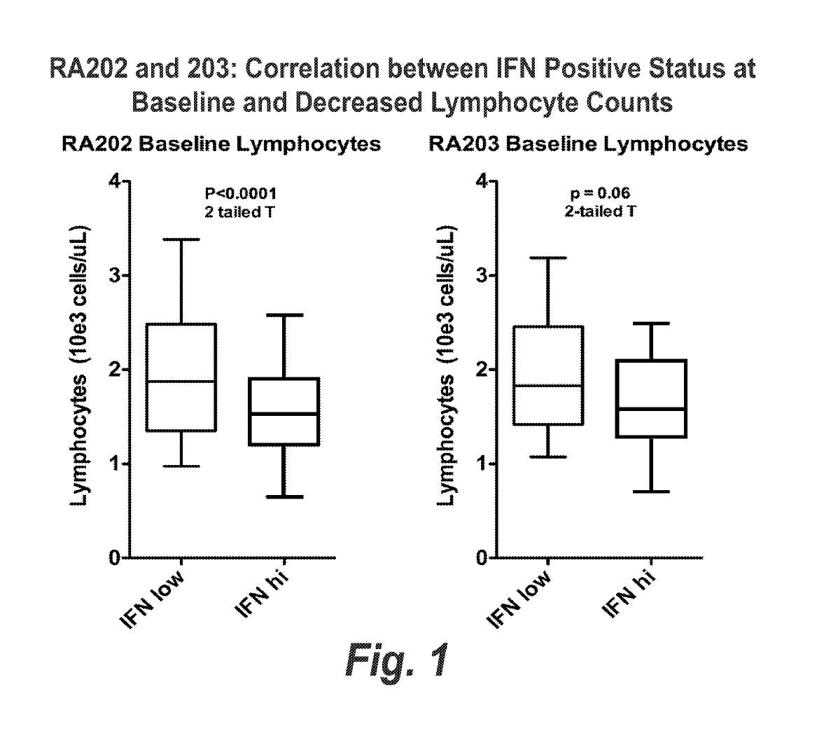

FIG. 2 shows that baseline white blood cell populations present in IFN+ rheumatoid arthritis patients (left and right panels) resemble those present in SLE patients (center panel).

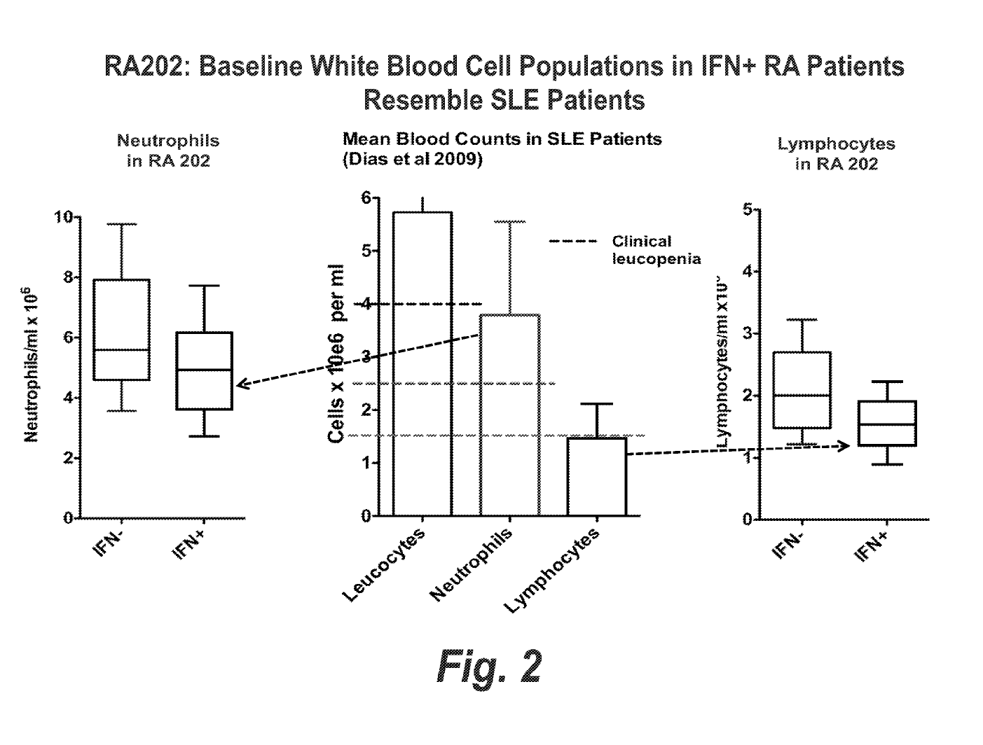

FIG. 3 shows a correlation between IFN positive status in patients treated with placebo (dark lines) and decreased lymphocyte counts in two groups of RA patients. Data from patients treated with soluble LTβR (Bam) are shown in grey.

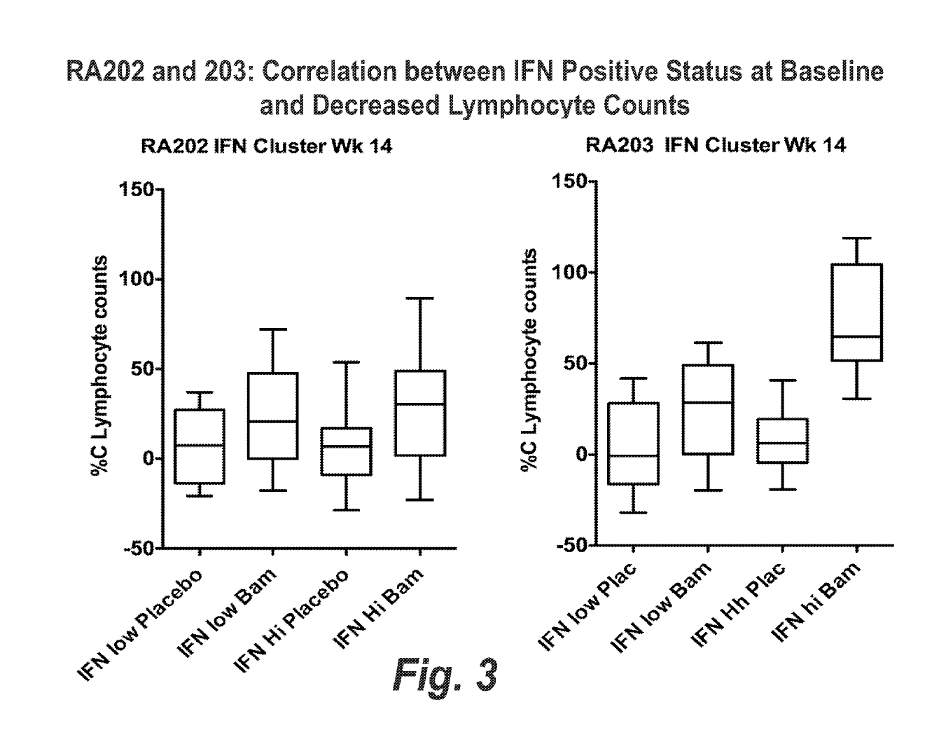

FIG. 4 shows that there is a correlation between IFN positive status at baseline and increased serum chemokine levels. Panel A shows levels of CXCL9, Panel B shows levels of CXCL10, and Panel C shows levels of CXCL13.

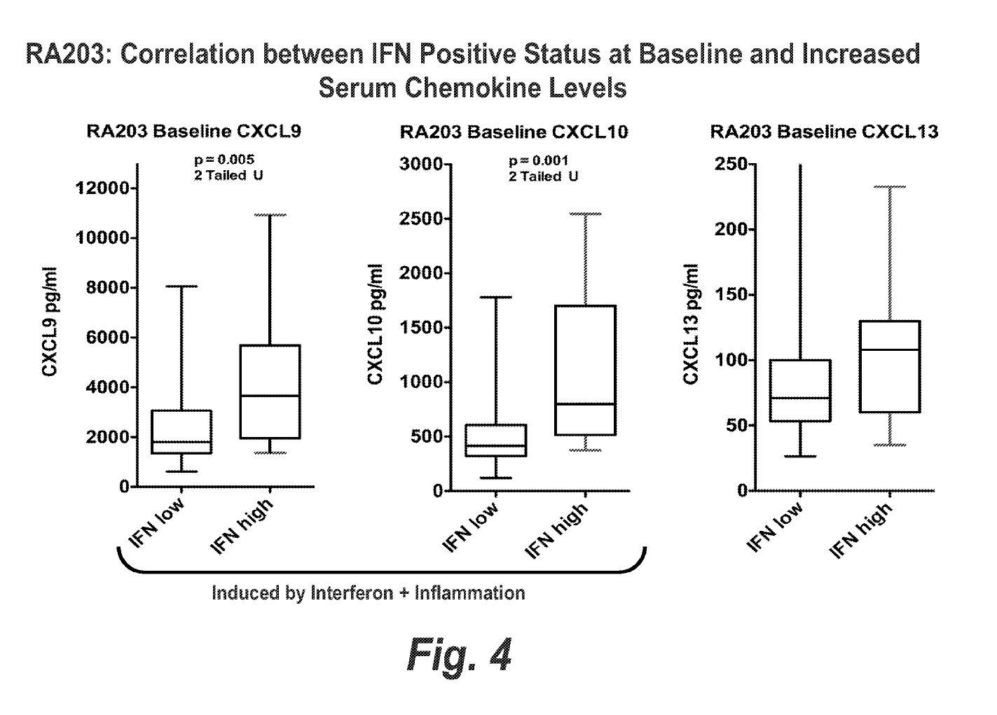

FIG. 5 shows that soluble LTβR reduces chemokine levels (here CXCL9) in IFN+ patients.

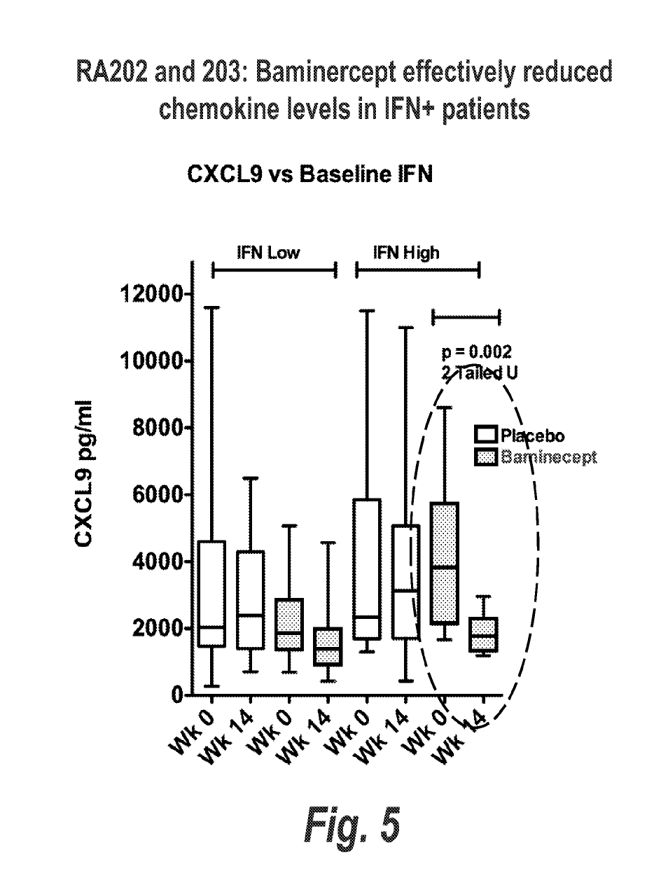

FIG. 6 shows that there is a correlation between IFN positive status at baseline and decreased swollen joint counts (SJC28). Data for patients receiving placebo are in dark lines and for patients receiving soluble LTβR are in grey.

FIG. 7 shows that there was a significant improvement in both the RA202 and the RA203 studies when looking at SJC28 data at week 14. Data for patients receiving placebo are in dark lines and for patients receiving soluble LTβR are in grey. The RA202 group contained Methotrexate inadequate responders and the RA203 group contained TNF inadequate responders.

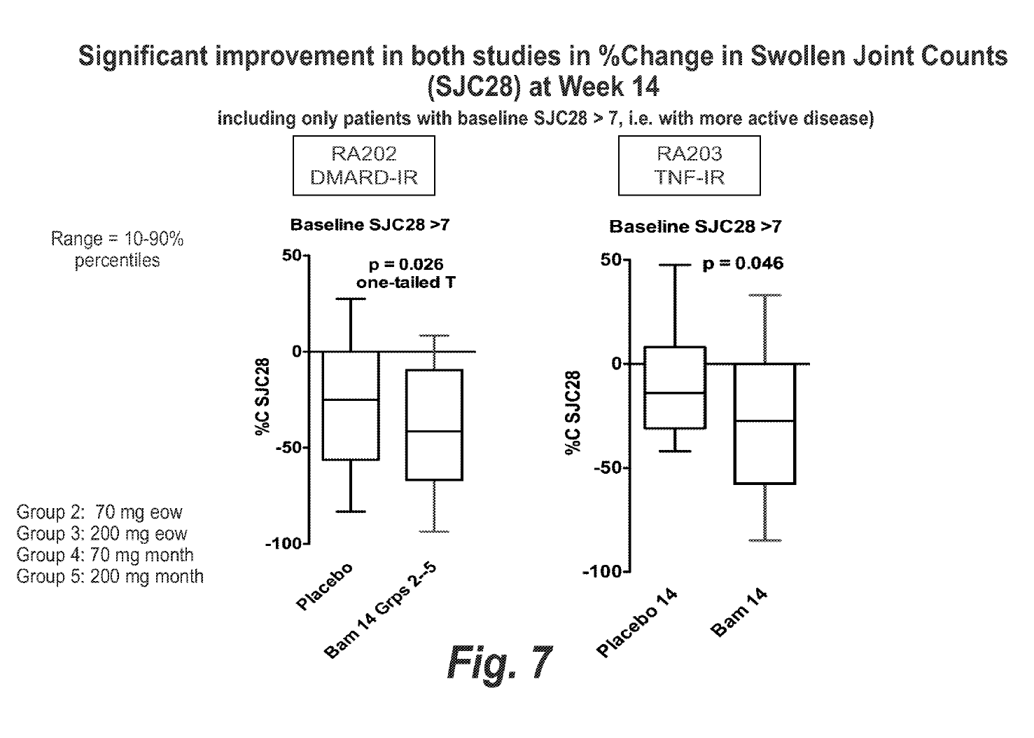

FIG. 8 shows that soluble LTβR reduced the IFN signature in TNF inadequate responders. Data for patients receiving placebo are in dark lines and for patients receiving soluble LTβR are in grey.

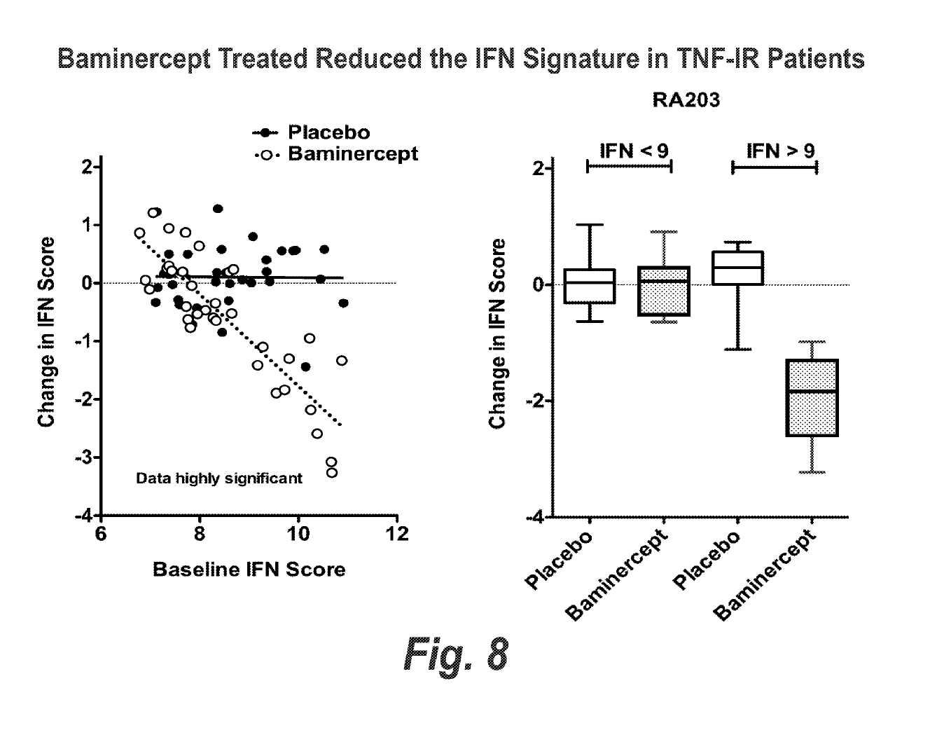

FIG. 9 shows that soluble LTβR had slight effects on the IFN signature in Methotrexate inadequate responders.

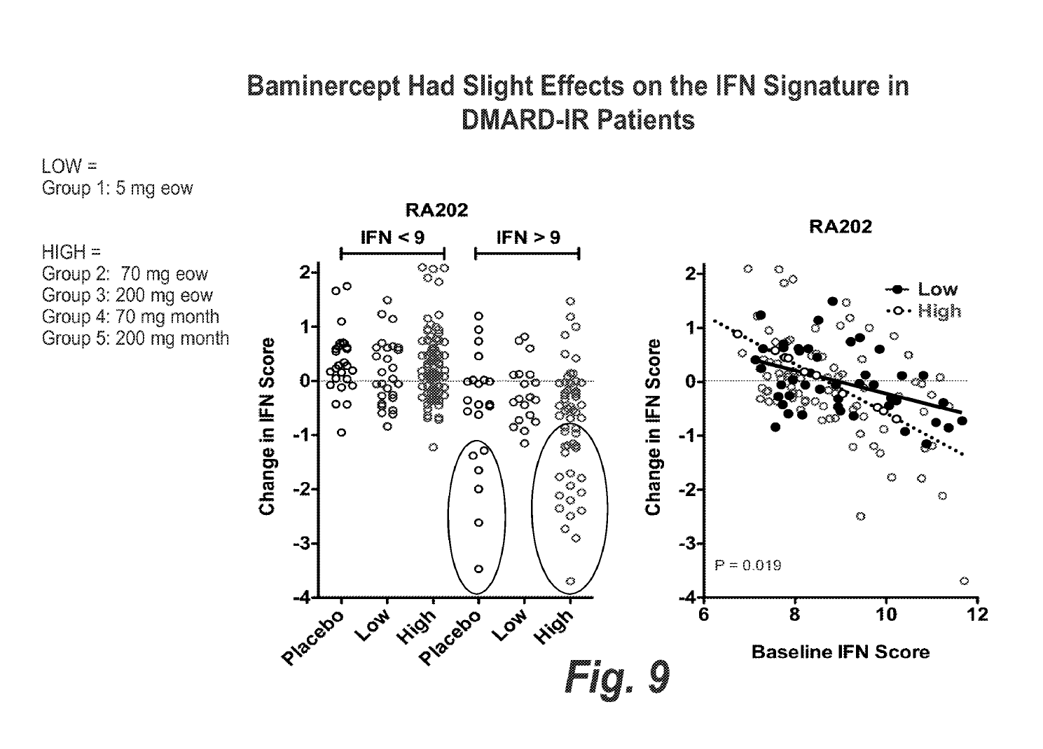

FIG. 10 shows that soluble LTβR reduced the IFN signature in RA patients. For these data, all treated RA patients were pooled.

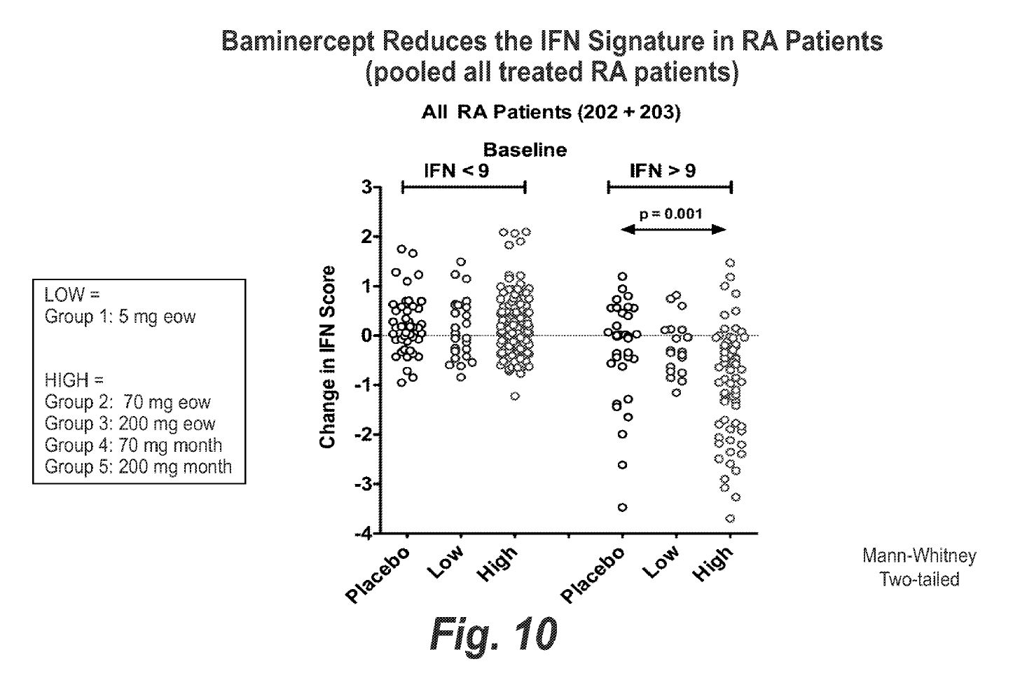

FIG. 11 shows the baseline IFN signatures for patients in the RA203 group (115 patients total). Twenty one out of 115 or 18% of patients have a strong signature. An additional 12 patients have a weaker signature for a total of 23% of patients. The list of genes whose expression was measured appears on the right and includes OAS3, HERC5, OAS1, TIMM10, RSAD2, IFI44L, IFI44, IFI6, IFIT3, RSAD2, MX1, DDX58, ISG15, UBE2L6, BATF2, and LIPA.

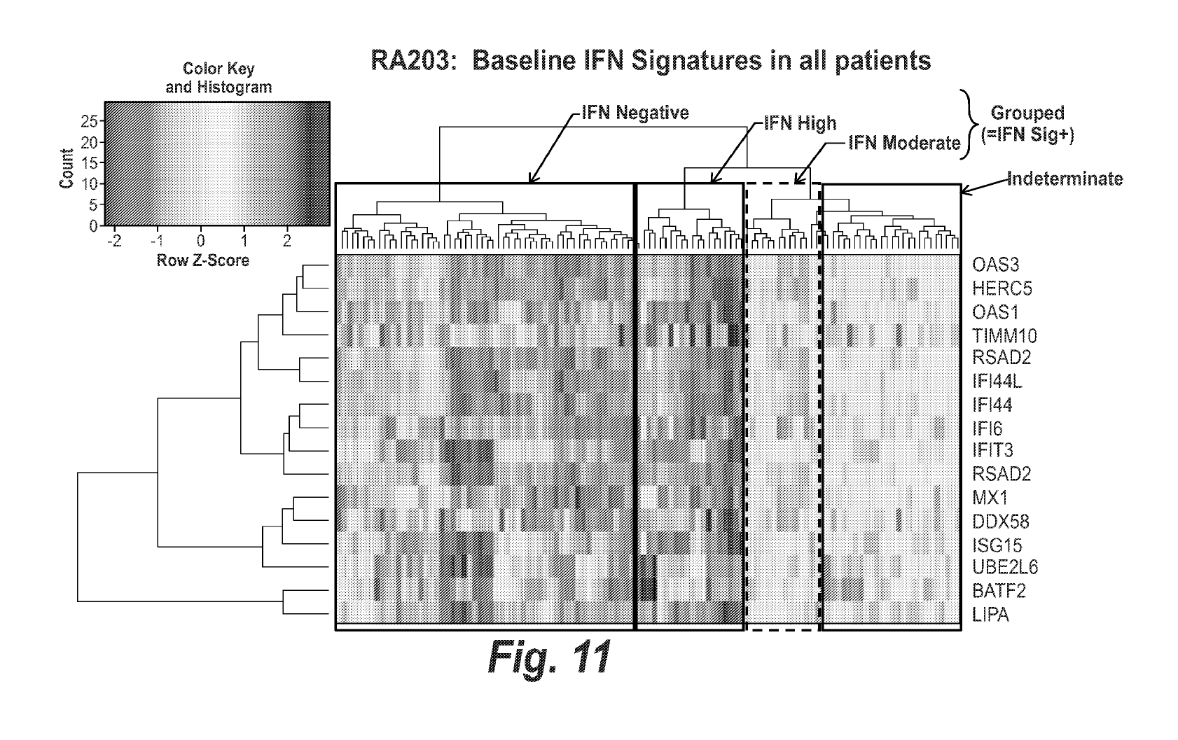

FIG. 12 shows the baseline IFN signatures for patients in the RA202 group. The list of genes whose expression was measured appears on the right and includes IFIT5, GBP, OASL, IFIT2, IFIT1, IFI44, ISG15, IFIT3, MXI, OAS3, IFI441, OAS1, SERPING1, and IRF5.

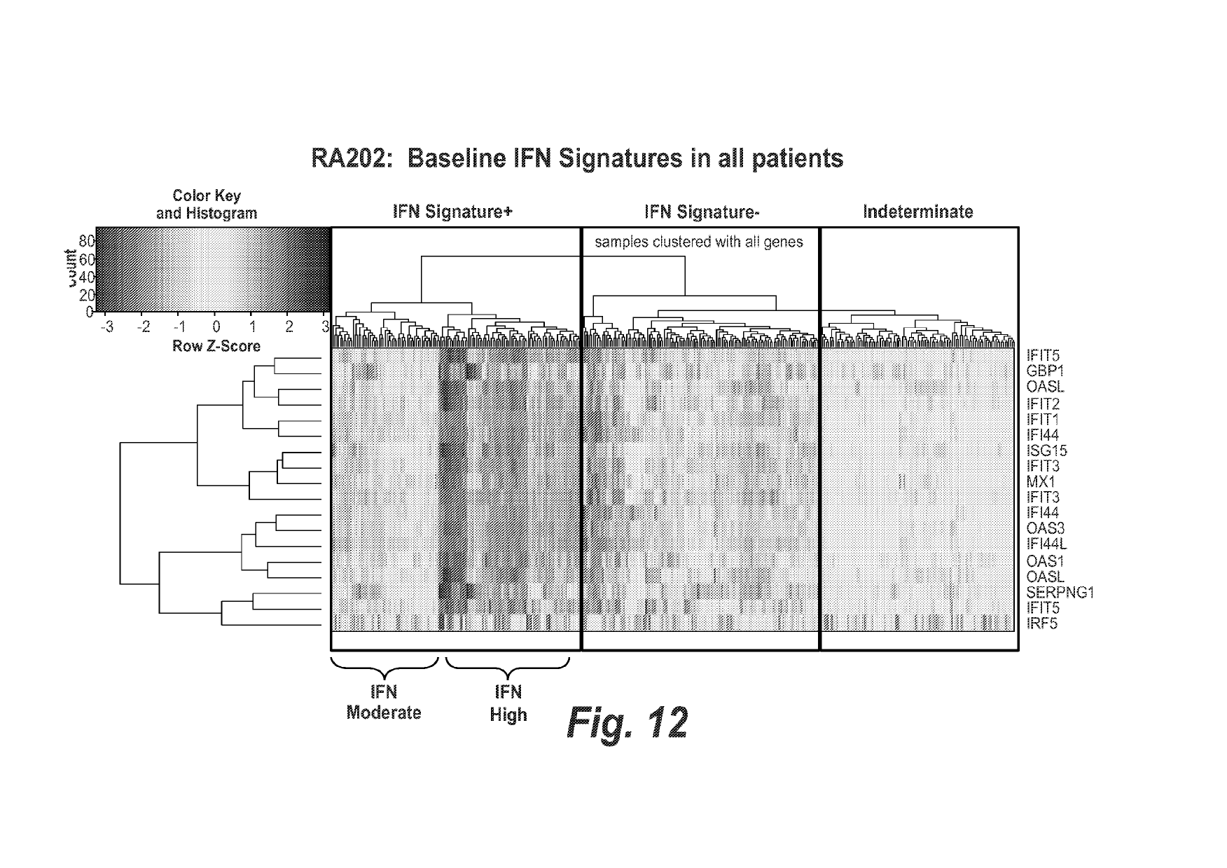

FIG. 13 shows that in the RA202 group, IFN signature status at entry was associated with slightly elevated ESR (erythrocyte sedimentation rate) and CRP (c-reactive protein). Low IFN signature patient data are shown in black and high IFN signature date are shown in grey.

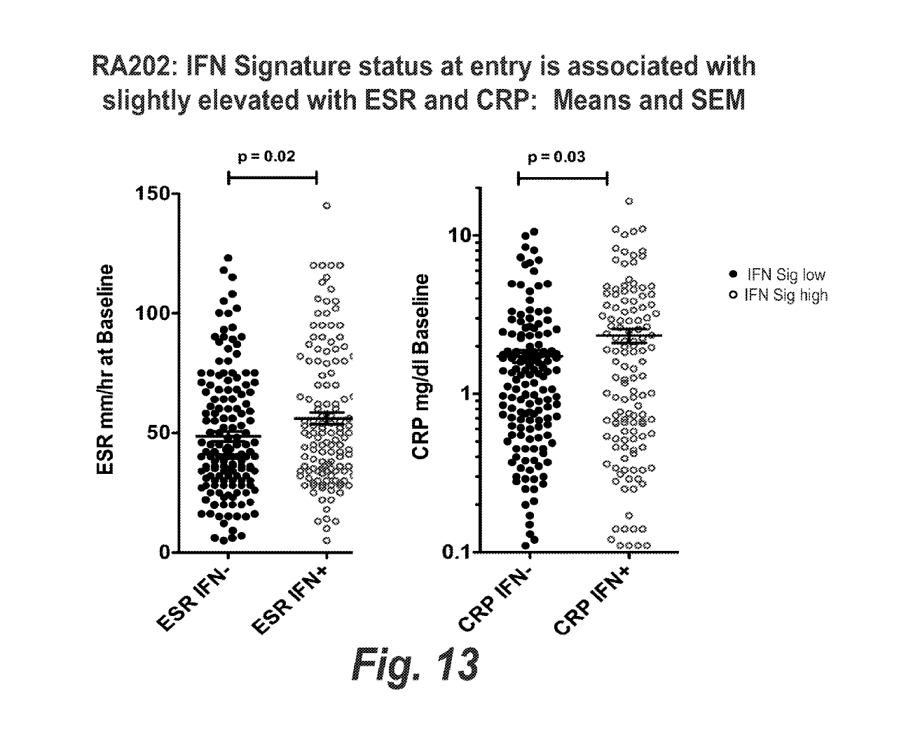

FIG. 14 shows that LTβRIg treatment reduces serum homeostatic chemokine levels in RA patients to approximately normal levels. 114 TNR-IR patients were randomized 2:1. Placebo data are in black and LTβRIg (200 mg every other week (eow)) are shown in grey.

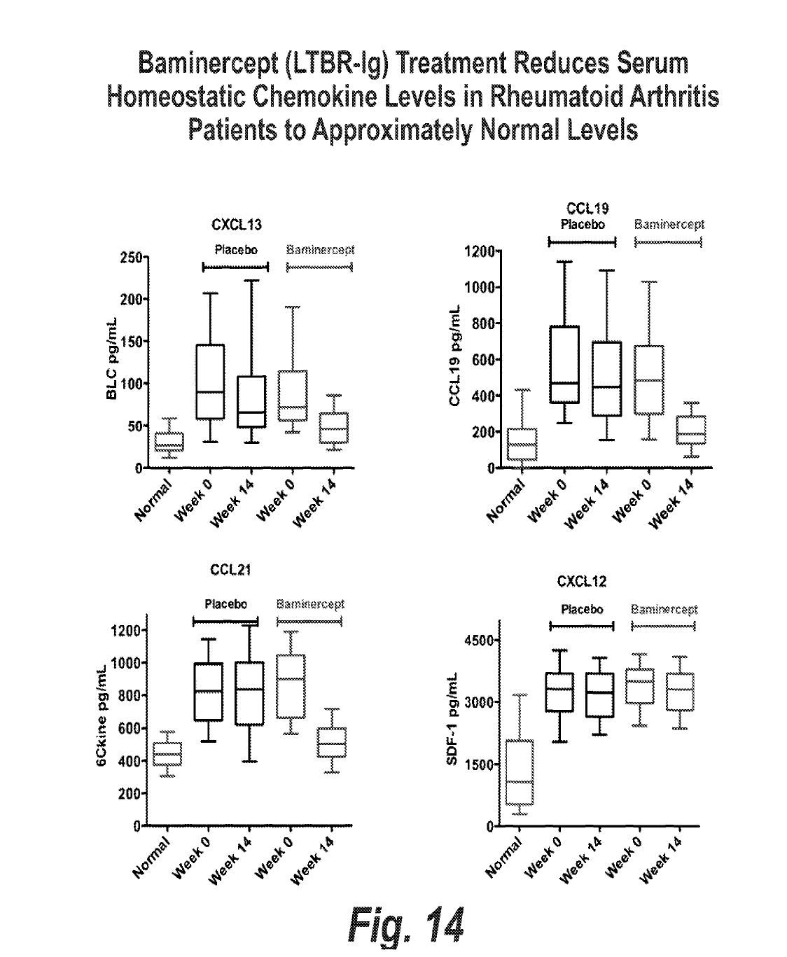

FIG. 15 shows the effects of LTβRIg on chemokines in the RA203 group. CXCL9 was downmodulated, CXCL10 reductions were not statistically significant, however, CXCL10 high patients appear to be normalized. Placebo data are in black and LTβRIg are shown in grey.

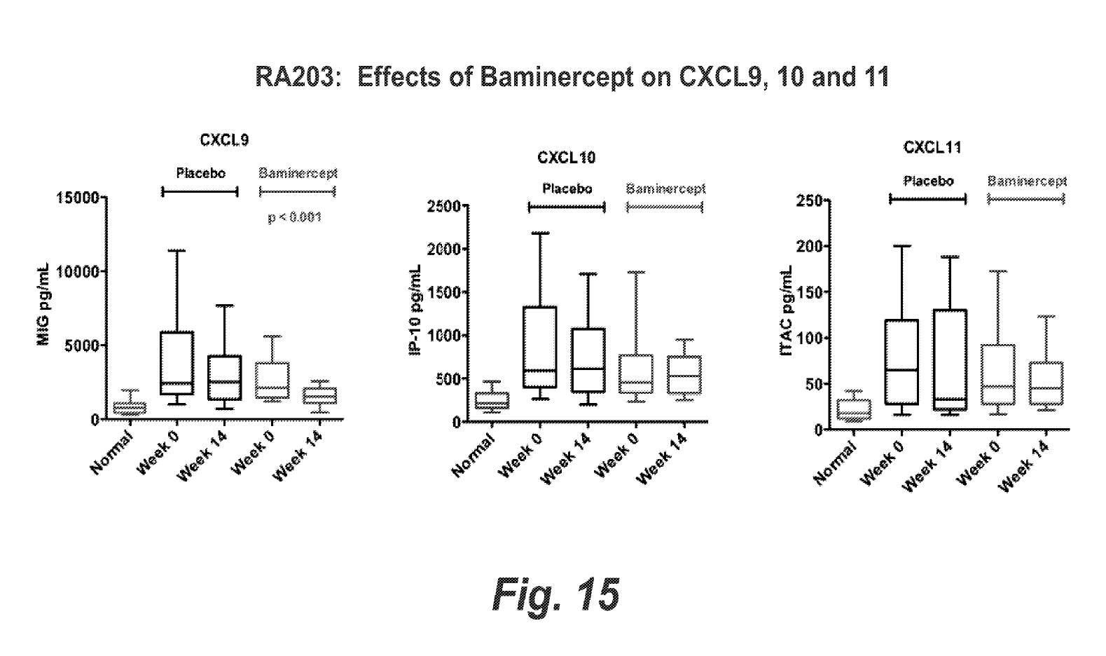

FIG. 16 shows changes in a type I IFN signature after 6 and 14 weeks of LTβRIg treatment in the RA203 group. The list of genes whose expression was measured appears on the right and includes IFI44, IFI6, RSAD2, IFIT3, TIMM10, OAS1, OAS3, HERC5, RSAD2, IFI44L, BATF2, LIPA, MXI, DDX58, ISG15, and UBE2L6.

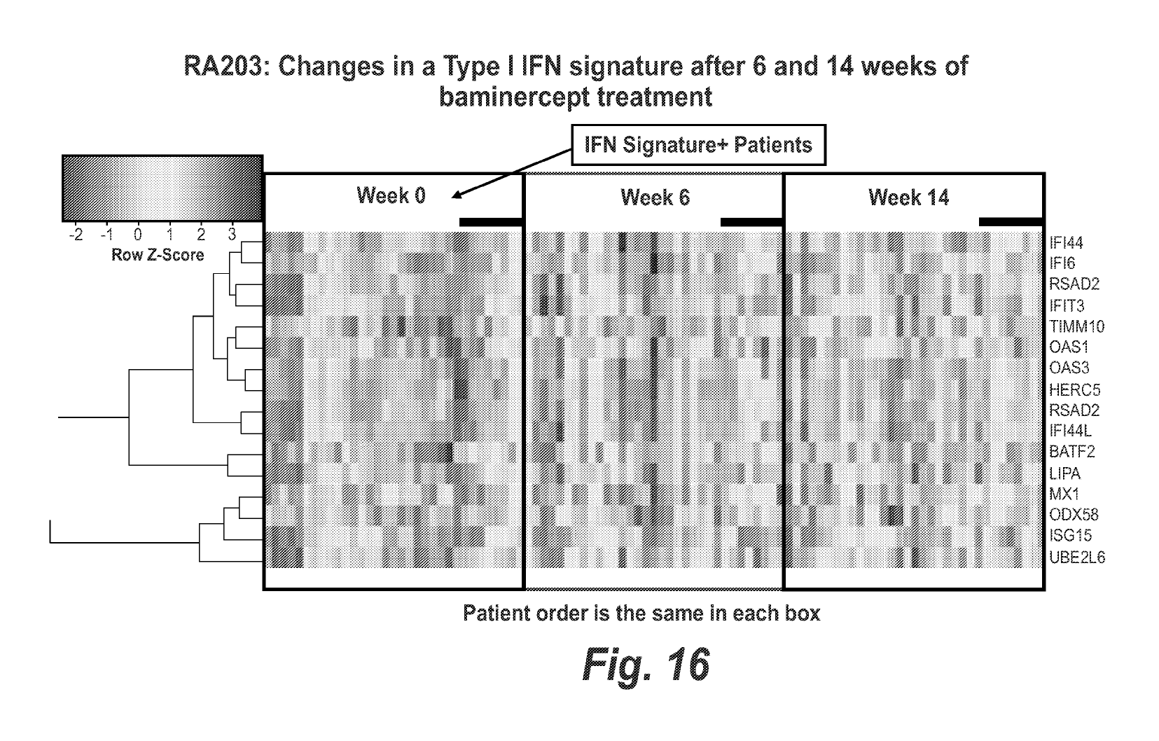

FIG. 17 shows that there were no changes in a type I IFN signature after 6 and 14 weeks in placebo treated patients. The list of genes whose expression was measured appears on the right and includes OAS3, OAS 1, TIMM10, HERC5, RSAD2, IFI44L, IFI5, IFI44, RSAD2, IFIT3, MXI, DDX58, ISG15, UBE2L5, BATF2, and LIPA.

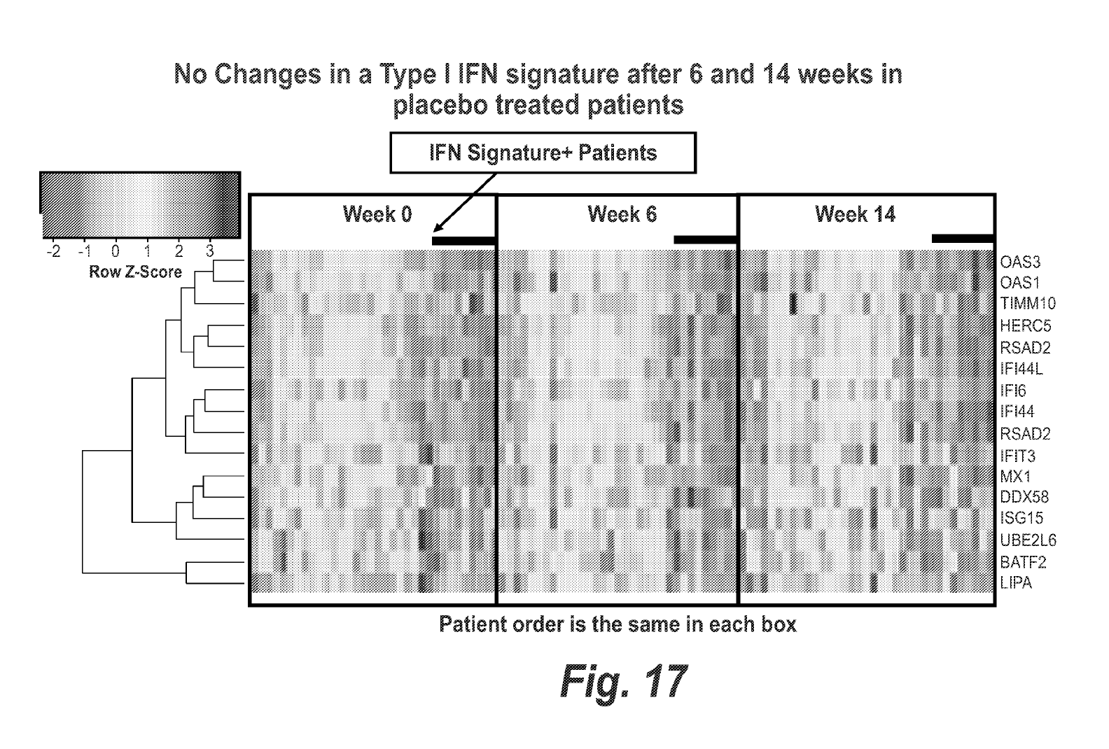

FIG. 18 shows a schematic of the transcriptional profiling study in the RA203 group.

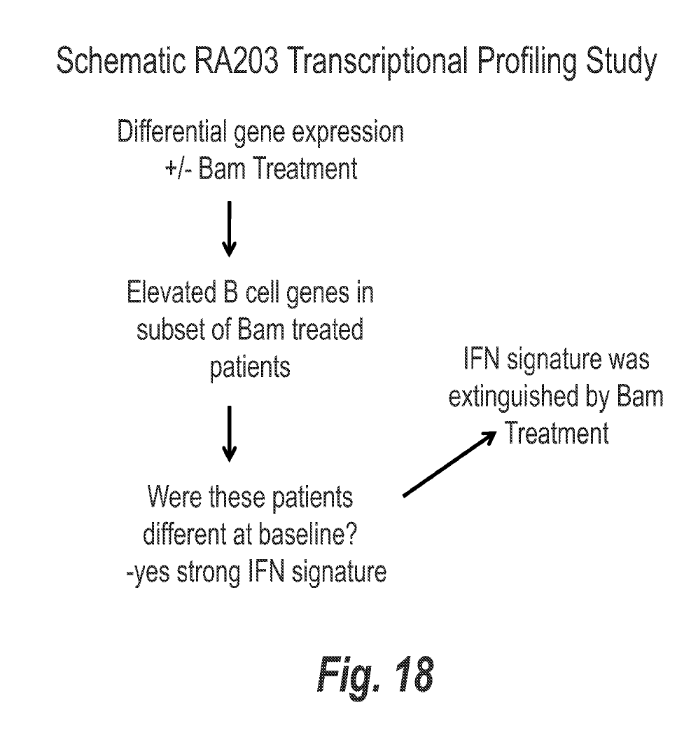

FIG. 19 shows that the LTβR induced change in lymphocyte counts is greater in baseline IFN signature high patients.

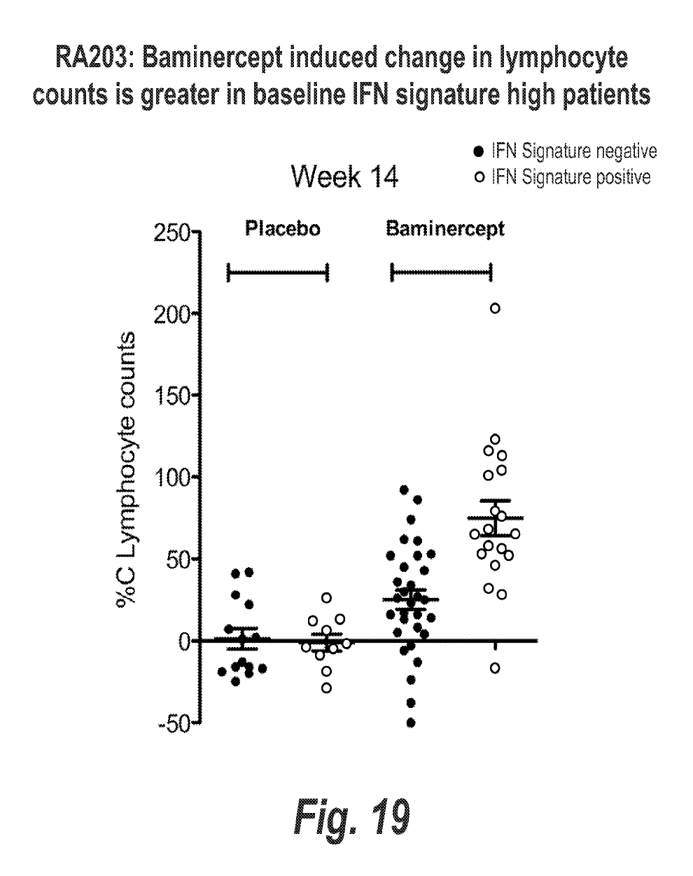

### DETAILED DESCRIPTION OF THE INVENTION

The invention provides, inter alia, methods for predicting therapeutic responsiveness (and thereby optimizing the efficacy of treatment) to an agent that inhibits signaling via LTβR in a subject having an autoimmune disorder; methods for selecting and/or administering a treatment regimen with an agent that inhibits signaling via LTβR based on the presence or absence of an interferon-inducible gene signature in the subject; and kits for selecting and/or treating subjects having an autoimmune disorder with an agent that inhibits LTβR signaling. Methods of treating selected patients are also provided. The invention is based, at least in part, on the observation that the presence of increased or decreased levels of IFN or a marker thereof (e.g., an increased interferon signature pattern) in a subject suffering from an autoimmune disorder is associated with increased or decreased responsiveness to therapy with an agent that inhibits LTβR signaling, respectively. More specifically, data from patients have been analyzed and these data show that subjects having increased expression of certain genes induced by IFN (e.g., IFNα, β, and/or γ) are more responsive to treatment with agents that inhibit signaling via LTβR, while subjects that do not have increased expression of these genes are not as responsive to such therapy.

Accordingly, the level of expression of IFN or one or more downstream markers thereof can be assessed in subjects having an autoimmune disease in order to predict responsiveness of a subject to therapy with an agent that inhibits signaling via LTβR, and/or to aid in the selection of an appropriate treatment regimen, and/or to provide therapy to such subjects.

### I. DEFINITIONS

In order that the present invention may be more readily understood, certain terms are first defined. Additional definitions are set forth throughout the detailed description.

An interferon-inducible gene expression signature refers to an increase in the expression of at least one IFN-inducible gene (i.e., a gene induced by IFNα, β, and/or γ) in a subject as compared to an appropriate control. For example, in one embodiment, such a signature is present in a subject having an autoimmune disorder, or at least one symptom thereof, and is not present in a control (e.g., a subject not having such a disorder or the same subject prior to onset of the symptom or disorder).

As used herein the term “increase” in IFN levels or a marker thereof refers to the presence of a higher level of IFN or a marker thereof as compared to an appropriate control. Levels of IFN or downstream markers of IFN can be increased or higher, e.g., relative to a subject that does not have an autoimmune disease (e.g., a normal subject) or relative to a subject that has an autoimmune disease, but has IFN levels (or levels of a marker thereof) which are not increased above those in a normal subject or relative to a subject that has an autoimmune disease, but did not respond well to an agent that blocks signaling vial LTβR. In one embodiment, an increase in IFN levels or a marker thereof is statistically significant using an appropriate statistical test. In another embodiment, an “increase” meets one or more of the following criteria: an increase of at least about 1.5-fold (e.g., 1.3 fold, 1.4 fold, 1.5 fold or greater) as compared to an appropriate control.

As used herein, the term “increase in expression” refers to an increase in expression of a gene as compared to an appropriate control. In one embodiment an increase in expression is statistically significant using an appropriate statistical test. In another embodiment, an “increase in expression” meets one or more of the following criteria: an increase in expression of at least about 1.5-fold (e.g., 1.3 fold, 1.4 fold, 1.5 fold or greater); an increase in expression of at least about 100 AD units (e.g., 90 units, 95 units, 96 units, 97 units, 98 units, 99 units or greater) or; or a statistically significant increase in expression (e.g., having P value of 0.05 or less for example as measured by an appropriate statistical test) as compared to an appropriate control.

As demonstrated herein, increased expression of IFN or a marker thereof (e.g., an autoantibody associated with increased IFN levels, a chemokine, and/or at least one IFN-inducible gene) in a subject is associated with increased responsiveness to therapy with an agent that inhibits signaling via LTβR in subjects having autoimmune disorders. The methods, compositions and kits of the present invention therefore provide a means for selecting patients having autoimmune disorders that are more likely to respond to LTβR blockade, thereby enhancing the therapeutic efficacy of such treatment.

The term “predicting responsiveness” to treatment with an agent that inhibits signaling via LTβR, as used herein, refers to an ability to assess the likelihood that treatment of a subject with an agent that inhibits signaling via LTβR will or will not be more clinically effective (e.g., provide an increased measurable benefit to) in the subject. Subjects having an increased IFN or a marker thereof can then be selected for treatment with an agent that inhibits signaling via LTβR. The ability to assess the likelihood that treatment will or will not be more clinically effective typically is exercised before treatment with the agent that inhibits signaling via LTβR is initiated. However, it is also possible that the ability to assess the likelihood that treatment will or will not be clinically effective can be exercised after treatment has begun to aid in optimizing treatment protocols. In one embodiment, a subject can be tested after treatment with a different agent (e.g., one that does not inhibit signaling via LTβR) has been initiated.

As used herein, the term “subject” includes humans and non-human animals amenable to therapy with an agent that inhibits signaling via LTβR, e.g. preferably mammals, such as non-human primates, sheep, dogs, cats, horses and cows and other domesticated mammals.

As used herein, the term “subject having an autoimmune disorder” refers to a subject having a form of autoimmune diseases or disorders, e.g., whether mediated by T cells or B cells or both (e.g., a subject having one or more sign or symptom thereof).

As used herein, the term “biological sample” refers to a sample obtained from a subject in which gene transcription can be detected, e.g., bodily fluids, cells, tissues, or isolated genetic material.

As used herein, the term “treatment regimen” refers to one or more parameters selected for the treatment of a subject, e.g., with an agent that inhibits signaling via LTβR, which parameters can include, but are not necessarily limited to, the subset of patients to which treatment shall be administered, the type of agent chosen for administration, the dosage, the formulation, the route of administration and the frequency of administration.

Various aspects of the invention are described in further detail in the following subsections.

### II. METHODS OF PREDICTING RESPONSIVENESS TO AGENTS THAT INHIBIT SIGNALING VIA LTβR

The invention is based, at least in part, on the observation that subjects having increased levels of IFN (or markers which indicate increased levels of IFN) are more responsive to treatment with agents that inhibit signaling via LTβR, while subjects that do not demonstrate increased levels of IFN (or markers which indicate increased levels of IFN) are not as responsive to such therapy.

Therefore, determining whether IFN levels are increased (or determining whether downstream markers of increased levels of IFN are present) in a subject is a useful method of selecting subjects that will optimally respond to treatment with agents that inhibit signaling via LTβR and/or selecting treatment protocols for those subjects. Various art-recognized methods of detecting increased IFN levels in a subject are set forth below.

A. Reporter Cell Lines

In one embodiment, a reporter cell line can be used as a reagent to measure levels of IFN in a subject as is known in the art. For example, in one embodiment, cells which express genes responsive to IFN, e.g., WISH cells (available from ATCC as catalog number CCC125), can be used as reporter cells and can be contacted with a biological sample from a subject (e.g., serum) for an appropriate period of time. The reporter cells can then be analyzed for the presence or absence of IFN-induced gene transcripts, e.g., IFIT1, MX1, PKR, and/or one or more of the IFN-inducible genes described herein, as a means of detecting the presence of IFN in the biological sample. Such assays are described, e.g., in Weckerle et al. 2011. Arthritis & Rheumatism. 63:1044. Levels of gene transcripts can be measured using methods known in the art, e.g., as set forth in more detail below.

B. Detection of Autoantibodies

In one embodiment, certain autoantibodies can be used as markers of increased levels of IFN in a subject. Certain autoantibodies, e.g., anti-Ro, anti-double-stranded DNA, anti-La, anti-Sm, and ant-RNP antibodies have been associated with increased levels of IFN. (See, e.g., Weckerle et al. 2011. Arthritis & Rheumatism. 63:1044). Levels of these antibodies can be measured in a biological sample from a subject using reagents and methods known in the art and increased levels of these antibodies can be used as a marker of increased IFN in a subject.

C. Detection of IFN-Inducible Genes

In one embodiment, the expression of one or more genes induced by IFN in cells of a subject is measured as a marker of increased IFN expression. In one embodiment, the increased expression of the one or more genes (e.g., whether or not an interferon-inducible gene expression signature is present) is indicative of increase levels of IFN being present in the subject.

The compilation of the expression levels of all of the mRNA transcripts sampled at any given time point in any given sample comprises the gene expression profile or “signature.” Methods of gene expression profiling are known in the art. In particular, certain genes have been found to be “IFN signatures” in certain patients with autoimmunity (see, e.g., Baechler et al. Immunological Reviews 2006. 210:120-137 and the references cited therein and see also Yao et al. 2009. Human Genomics and Proteomics, Article ID 374312 and the references cited therein).

Exemplary IFN-induced genes that can be tested to determine whether a subject has elevated levels of at least one IFN-inducible gene include at least one of: AGRN, ANKRD22, APOL6, ATF3, BATF2, BST2, C18orf49; C1QB, CCL23, CEACAM1, CHURC1, DDX58, DHRS9, EPST11, ETV7, FBX06, FCGR1A, FCGR1B, FER1L3, FLJ20035, FLJ42418, FRMD3, GBP1, GBP4, GBP5, GRAMD1B, H19, HERC5, HERC6, IFI27, IFI35, IFI44, IFI44L, IFI6, IFIH1, IFIT1, IFIT2, IFIT3, IFIT5, INDO, ISG15, KIAA1618, KLHL18, LAMP3, LAP3, LHFPL2, LILRA3, LIPA, LOC129607, LOC151146, LOC26010, LOC440836, LOC729936, LY6E, MOV10, MX1, NA, NAPA, NRN1, OAS1, OAS2, OAS3, OAS1, OR52K3P, PAM, PARP14, PARP9, PLSCR1, PML, PNPT1, PRIC285, RHOT1, RNF213, RSAD2, RTP4, SAMD4A, SAMD9, SAMD9L, SAMHD1, SCO2, SERPING1, SIGLEC1, STAT1, STAT2, TIMM10, NFAIP6, TRIM6, UBE2L6, USP18, WARS, WDYF1, XAF1, ZBP1, and ZCCHC2. In another embodiment, IFN-inducible genes include at least one of: APOL6, EPST11, GBP1, IFI35, IFI44, IFI44L, IFI6, IFIT1, IFIT3, IFI5, IFIT5, LIPA, OAS1, OAS2, OAS3, SERPING1, HERC5, TIMM10, RSDA2, ISG15, MXI, DDX58, DDX58, UBE2L6, BATF2, and XAF1. In another embodiment, IFN-inducible genes include at least one of: OAS3, HERC5, OAS1, TIMM10, RSDA2, IFI44L, IFI44, IFI6, IFIT3, ISG15, MXI, DDX58, DDX58, UBE2L6, BATF2, and LIPA. In another embodiment, IFN-inducible genes include at least one of: IFI44, IFI6, RSAD2, IFIT3, TIMM10, OAS1, OAS3, HERC5, RSAD2, IFI44L, BATF2, LIPA, MXI, DDX58, ISG15, and UBE2L6. In another embodiment, IFN-inducible genes include at least one of: OAS3, OAS1, TIMM10, HERC5, RSAD2, IFI44L, IFI5, IFI44, RSAD2, IFIT3, MXI, DDX58, DDX58, ISG15, UBE2L5, BATF2, and LIPA. IFI44, IFI6, RSAD2, IFIT3, TIMM10, OAS1, OAS3, HERC5, RSAD2, IFI44L, BATF2, LIPA, MXI, DDX58, ISG15, and UBE2L6. In another embodiment, IFN-inducible genes include at least one of: OAS3, OAS1, TIMM10, HERC5, RSAD2, IFI44L, IFI5, IFI44, RSAD2, IFIT3, MXI, DDX58, ISG15, UBE2L5, BATF2, and LIPA. Further information on these genes and others that are known to be induced by IFN and which can be used in the claimed methods can be found in the art (see, e.g., Yao et al. 2009. Human Genomics and Proteomics Article ID 374312; Tan et al. 2006 Rheumatology 45:694-702; Baecheler et al. 2003 PNAS USA 100:2610; Bennett et al. 2003. J. Exp. Med. 197:711; Yao et al. 2010. Arthritis Res. Therapy; Baechler et al. Immunological Reviews 2006. 210:120-137 and the references cited therein and see also Yao et al. 2009. Human Genomics and Proteomics, Article ID 374312 and the references cited therein.

### Methods of Measuring Expression of IFN-Induced Genes

To determine whether a subject has increased expression of one or more genes induced by IFN, a biological sample is obtained from the subject. For example, cells or tissue can be obtained. In one embodiment, bodily fluid samples that contain cells, such as blood, urine, semen, or saliva can be obtained. Biological samples may be obtained from a subject by a variety of techniques including, for example, a biopsy or by scraping or swabbing an area or by using a needle to aspirate. Methods for collecting various biological samples are well known in the art.

Preferably, prior to obtaining a biological sample, a subject will refrain from taking medications which can reduce immune responses (in particular IFN signatures), such as steroids.

In one embodiment, a sample can be transformed or manipulated prior to analysis by isolating genetic material from cells. Genetic material suitable for analysis can be derived from a variety of sources. For example, nucleic acid molecules (e.g., mRNA or DNA) can be isolated from cells or tissues using standard methods.

Various detection methods known in the art can be used to detect the level of expression of one or more genes present in a cell in a biological sample (e.g., in intact cells or in extracted nucleic acid). In one embodiment, the level of RNA transcripts or nucleic acid molecules derived therefrom can be measured. In another embodiment, the level of protein expressed can be detected. Alternatively, in another embodiment, protein activity can be measured.

In one embodiment, detection methods physically alter (i.e., transform) the nucleic acid molecule or protein molecule being tested into a detectable composition by the addition of a reagent (e.g., that is detectable on its own or which facilitates or allows for detection when combined with a component (e.g., a nucleic acid molecule or protein) present in the biological sample. Detection methods can measure the level of expression of a gene directly (e.g., by looking at nucleic acid molecules) or by looking at the protein specified by a nucleic acid molecule sequence. In another embodiment, detection methods can be indirect, e.g., can measure the activity of a protein.

In one embodiment, the method of detecting the level of expression of a gene in a biologic sample involves transformation of the sample into an altered form which can be detected using a readout detectable by eye or with the aid of a computer. In one embodiment, a computer also can be used to assist in detecting or quantitating the level of the detectable composition.

Exemplary means of manipulating biological samples into a form in which the expression of genes can be detected include making cell lysates or extracting nucleic acid molecules from a cell. Such methods are discussed in more detail below.

Nucleic Acid Extraction

Nucleic acids, such as RNA, may be isolated and purified from cells, tissues or fluids of a patient using readily-available and well-known procedures. One of skill in the art will appreciate that in order to measure the transcription level (and thereby the expression level) of a gene or genes, it is desirable to provide a nucleic acid sample comprising mRNA transcript(s) of the gene or genes, or nucleic acids derived from the mRNA transcript(s). As used herein, a nucleic acid molecule derived from an mRNA transcript refers to a nucleic acid molecule for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, suitable samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

In one embodiment, a nucleic acid sample is the total mRNA isolated from a biological sample. Methods of isolating total mRNA are well known to those of skill in the art. For example, methods of isolation and purification of nucleic acids are described in detail in Chapter 3 of Laboratory Techniques in Biochemistry and Molecular Biology:

Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization with Nucleic Acid Probes, Part I. Theory and Nucleic Acid Preparation, P. Tijssen, ed. Elsevier, N.Y. (1993)).

In one embodiment, RNA may be preferentially obtained from a nucleic acid mix using any of a variety of standard procedures (see, e.g., RNA Isolation Strategies, pp. 55-104, in RNA Methodologies, A laboratory guide for isolation and characterization, 2nd edition, 1998, Robert E. Farrell, Jr., Ed., Academic Press). Additionally, RNA isolation systems/kits are available from numerous commercial vendors, such as the RNAqueous™, Phenol-free Total RNA Isolation Kit offered by Ambion (Austin, Tex.) or the PicoPure RNA Isolation kit offered by Arcturus Bioscience (Mountainview, Calif.).

In certain embodiments, for example, RNA may be extracted from biological samples using methods known in the art, e.g., the PicoPure RNA Isolation kit. The quality of captured RNA is, preferably, examined following extraction. The quality of isolated RNA may be measured using well-known procedures, such as with an Agilent 2100 Bioanalyzer and RNA 6000 Pico LabChips (Agilent Technologies, Palo Alto, Calif.).

In one embodiment of the present invention, the isolated RNA is amplified for analysis (e.g., prior to gene expression profiling or other nucleic acid analysis described herein). In one embodiment, quantitative PCR can be used to measure expression levels. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. In one embodiment, an array or microarray may include probes specific to the internal standard for quantification of the amplified nucleic acid.

Those of ordinary skill in the art will appreciate that RNA amplification (and, optionally, labeling) may be carried out using commercially-available kits and/or well-known procedures. For example, RNA amplification may be carried out using a RiboAmp RNA Amplification Kit (Arcturus Bioscience, Mountainview, Calif.). Following such amplification step, the quality of the amplified RNA (and/or DNA) is, preferably, examined to determine quality. In one embodiment, expression levels of one or more genes can be quantitated using qualitative real-time RT-PCR using methods known in the art.

In one embodiment, the amplified RNA (and/or DNA) may be labeled with detectable labels. Such labels include any composition detectable by spectroscopic, photochemical, biochemical, immunochemical, electrical, optical or chemical means. Exemplary labels include biotin for staining with labeled streptavidin conjugate, magnetic beads (e.g., Dynabeads™), fluorescent dyes (e.g., fluorescein, texas red, rhodamine, green fluorescent protein, and the like), radiolabels, enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

Gene Expression Profiling & Other Nucleic Acid Analysis

In one embodiment, an “expression profile” or “gene expression profile” comprises measurement of a plurality of mRNAs to indicate the relative expression or relative abundance of any particular transcript. The compilation of the expression levels of all of the mRNA transcripts sampled at any given time point in any given sample comprises the gene expression profile or “signature.” Methods of gene expression profiling are known in the art. In particular, IFN signatures have been identified in certain patients with autoimmunity and the methods of producing these profiles can be used in connection with the instant invention (see, e.g., Baechler et al. Immunological Reviews 2006. 210:120-137 and the references cited therein and see also Yao et al. 2009. Human Genomics and Proteomics, Article ID 374312 and the references cited therein).

Common methods of expression profiling employ arrays or microarrays. Such arrays employ oligonucleotides which are either synthesized directly or spotted onto a solid support (e.g., a glass slide or a filter) see e.g., De Risi et al. 1997 Science 278:680 and Alizadeh et al. 1998 J. Clin. Immunol. 18:373. The arrays are designed to measure the expression levels of the genes represented in the array based on the hybridization of test oligonucleotides prepared from the biological sample. Arrays can also be fabricated. Two commonly used array types are based on cDNA fragments or short oligonucleotides produced by photolithography or longer oligonucleotide probes produced by inkjet printing processes see, e.g., Pease et al. 1994. PNAS USA 91:5022. One embodiment of the invention involves monitoring gene expression by (1) providing a pool of target nucleic acids comprising RNA transcript(s) of one or more target gene(s), or nucleic acids derived from the RNA transcript(s); (2) hybridizing the nucleic acid sample to a array of probes (e.g., including control probes); and (3) detecting the hybridized nucleic acids and calculating a relative expression (transcription) level.

In one embodiment, where it is desired to quantify the transcription level (and thereby expression) of a one or more genes in a sample, the nucleic acid sample is one in which the concentration of the mRNA transcript(s) of the gene or genes, or the concentration of the nucleic acids derived from the mRNA transcript(s), is proportional to the transcription level (and therefore expression level) of that gene. Similarly, in one embodiment, the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Where more precise quantification is required, appropriate controls can be run to correct for variations introduced in sample preparation and hybridization. In addition, serial dilutions of “standard” target mRNAs can be used to prepare calibration curves according to methods well known to those of skill in the art. Of course, where simple detection of the presence or absence of a transcript is desired, no elaborate control or calibration is required.

Arrays

Arrays or microarrays may be purchased commercially from vendors such as Affymetrix (Santa Clara, Calif.) and Agilent Technologies (Santa Clara, Calif.). Alternatively, they may be made using known techniques. When making an array, oligonucleotides are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., 1995 (Quantitative monitoring of gene expression patterns with a complementary DNA array, Science 270:467-470). This method is especially useful for preparing arrays of cDNA. See also DeRisi et al., 1996 (Use of a cDNA array to analyze gene expression patterns in human cancer, Nature Genetics 14:457-460; Shalon et al., 1996, A DNA array system for analyzing complex DNA samples using two-color fluorescent probe hybridization, Genome Res. 6:639-645; and Schena et al., 1995, Parallel human genome analysis; array-based expression of 1000 genes, Proc. Natl. Acad. Sci. USA 93:10614-10619). Each of the aforementioned articles is incorporated by reference in its entirety.

Another method for making arrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Light-directed spatially addressable parallel chemical synthesis, Science 251:767-773; Pease et al., 1994, Light-directed oligonucleotide arrays for rapid DNA sequence analysis, Proc. Natl. Acad. Sci. USA 91:5022-5026; Lockhart et al., 1996, Expression monitoring by hybridization to high-density oligonucleotide arrays, Nature Biotech 14:1675; U.S. Pat. Nos. 5,578,832; 5,556,752; and 5,510,270, each of which is incorporated by reference in its entirety) or other methods for rapid synthesis and deposition of defined oligonucleotides (Blanchard et al., 1996, High-Density Oligonucleotide arrays, Biosensors & Bioelectronics 11: 687-90). When these methods are used, oligonucleotides (e.g., 20-mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs. Another preferred method of making arrays is by use of an inkjet printing process to synthesize oligonucleotides directly on a solid phase.

Other methods for making arrays; e.g., by masking (Maskos and Southern, 1992, Nuc. Acids Res. 20:1679-1684), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989, which is incorporated in its entirety), could be used, although, as will be recognized by those of skill in the art, small arrays will be preferred because hybridization volumes will be smaller.

In one embodiment, a array for use in detecting expression of at least one IFN-inducible gene comprises oligonucleotide probes having sequences complementary to particular subsequences of the genes whose expression they are designed to detect. Thus, the test probes are capable of specifically hybridizing to the target nucleic acid sequences.

The present invention measures expression of one or more genes which are induced by IFN. In one embodiment, the expression of at least one gene regulated by IFNα is measured. In one embodiment, expression of at least one IFN-inducible gene is measured. In another embodiment, expression of more than one, i.e., a plurality, of IFN-inducible genes is measured. It will be understood by those of skill in the art that in order to detect enhanced expression of at least one IFN-inducible gene, it may be desirable to measure the expression of more than one IFN-inducible gene as not all such genes may demonstrate increased expression levels. In one embodiment, expression of at least 2 IFN-inducible genes is measured. In one embodiment, expression of at least 3 IFN-inducible genes is measured. In one embodiment, expression of at least 4 IFN-inducible genes is measured. In one embodiment, expression of at least 5 IFN-inducible genes is measured. In one embodiment, expression of at least 6 IFN-inducible genes is measured.

In one embodiment, expression of at least 7 IFN-inducible genes is measured. In one embodiment, expression of at least 8 IFN-inducible genes is measured. In one embodiment, expression of at least 9 IFN-inducible genes is measured. In one embodiment, expression of at least 10 IFN-inducible genes is measured. In one embodiment, expression of at least 15 IFN-inducible genes is measured. In one embodiment, expression of at least 20 IFN-inducible genes is measured. In one embodiment, expression of at least 25 IFN-inducible genes is measured. In one embodiment, expression of at least 30 IFN-inducible genes is measured. In one embodiment, expression of between 1 and 100 IFN-inducible genes is measured.

Exemplary IFN-inducible genes and probes that can be used to detect them include: AGRN (212285_s_at), ANKRD22 (238439_at or 239196_at), APOL6 (1557236_at, 219716_at, or 241869_at), ATF3 (202672_sat), BATF2 228439_at), BST2 (201641_at), Cl8orf49 (232222_at); ClQB (202953_at), CCL23 (210549_s_at), CEACAM1 (206576_s_at or 211889_x_at), CHURC1 (226736_at), DDX58(222793_at or 218943_s_at), DHRS9 (219799_s_at, or 223952_x_at, or 224009_x_at), EPST11(235276_at, 239979_at, or 227609_at), ETV7 (221680_s_at or 224225_s_at), FBX06 (231769_at), FCGR1A (216950_s_at or 216951_at), FCGR1B (214511_x_at), FER1L3 (201798_s_at), FLJ20035 (218986_s_at), FLJ42418 (231455_at), FRMD3(229893_at), GBP1 (202270_at or 202269_x_at or 231577_s_at), GBP4 (235175_at or 235574_at), GBP5 (229625_at or 238581_at), GRAMD1B (212906_at), H19(224997_x_atH19), HERC5(219863_at), HERC6 (239988_at; 2022411_at), IFI27 (202411_at), IFI35 (209417_s_at), IFI44 (214453_s_at or 214059_at), IFI44L (204439_at), IFI6 (204415_at), IFIH1(219209_at), IFIT1 (203153_at), IFIT2 (226757_at or 217502_at), IFIT3 (229450_at or 204747_at), IFITS (203596_s_at or 203595_s_at), INDO (210029_at), ISG15 (205483_s_at), KIAA1618 (231956_at), KLHL18(1557165_s_a), LAMP3 (205569_at), LAP3 (217933_s_at), LHFPL2(212658_at), LILRA3 (206881_s_at), LIPA(236156_at), LOC129607 (226702_at), LOC151146 (241237_at), LOC26010 (222154_s_at), LOC440836(238327_at), LOC729936 (a570541_s_a), LY6E(202145_at), MOV10 (223849_s_at), MX1 (202086_at), NA (156175_at or 224348_s_at or 243754_at), NAPA (206491_s_at), NRN1(218625_at), OAS1 (202869_at or 205552_s_at), OAS2 (204972_s_at or 228607_at), OAS3 (218400_at), OASL (205660_at or 210797_s_at), OR52K3P (232829_at), PAM(214620_x_at or 242457_at or 202336_s_at), PARP14 (235157_at or 232610_at), PARP9 (227807_at), PLSCR1 (202430_s_at or 202446_s_at), PML (235508_at or 211012_s_at), PNPT1 (225291_at), PRIC285 (228230_at), RHOT1(230314_at), RNF213 (233880_at), RSAD2 (237538_at or 213797_at or 242625_at), RTP4 (219684_at), SAMD4A (212845_at or 215495_s_at), SAMD9 (219691_at), SAMD9L (230036_at or 226603_at or 235643_at or 243271_at), SAMHD1 (1559882_at), SCO2 (205241_at), SERPING1 (200986_at), SIGLEC1 (219519_s_at or 44673_at), STAT1 (209969_s_at or AFFX-HUMIS or 232375_at), STAT2 (205170_at), TINIM10 (218408_at or 1555764_s_a), TNFAIP6 (206025_s_at), TRIM6 (223599_at), UBE2L6 (201649_at), USP18 (219211_at), WARS (20628_s_at), WDYF1 (242390_at), XAF1 (228617_at or 206133_at or 242234_at), ZBP1 (208087_s_at), and ZCCHC2 (219062_s_at or 222816_s_at). Further information on these genes and others that are induced by IFN can be found in the art (see, e.g., Yao et al. 2009. Human Genomics and Proteomics Article ID 374312).

Additional nucleic acid molecules for detection of expression of these genes are known in the art or can readily be derived by one of ordinary skill in the art based on the nucleic acid sequences of the molecules.

In one embodiment, the assay is an assay based on detecting the level of gene transcription (e.g., an array or microarray or qPCR) and the level of transcription of a plurality of genes induced by IFN is detected. In one embodiment, the level of transcription of a plurality of genes induced by IFNα is detected. In one embodiment, the level of transcription of at least 3 genes induced by IFN is detected. In one embodiment, the level of transcription of at least 4 genes induced by IFN is detected. In one embodiment, the level of transcription of at least 5 genes induced by IFN is detected. In one embodiment, the level of transcription of at least 7 genes induced by IFN is detected. In another embodiment, the level of transcription of at least 10 genes induced by IFN is detected. In one embodiment, the level of transcription of at least 12 genes induced by IFN is detected. In another embodiment, the level of transcription of at least 15 genes induced by IFN is detected. In another embodiment, the level of transcription of at least 20 genes induced by IFN is detected. In one embodiment, the level of transcription of between 1 and 100 genes induced by IFN is detected.

It will be understood by those of skill in the art that the demonstration that subjects having increased expression of at least one gene inducible by IFN are more responsive to LTβR blockade means that although the genes listed above may be preferred, other IFN inducible genes known in the art (in particular those previously shown to be present at increased levels in subjects having IFN signatures, e.g., certain subjects having autoimmune disorders) may be substituted.

In one embodiment, an array for use with the instant invention will include one or more control probes. Exemplary control probes fall into three categories referred to herein as a) Normalization controls; b) Expression level controls; and c) Mismatch controls. In one embodiment, at least one no-template control can be included.

Normalization controls are oligonucleotide probes that are perfectly complementary to labeled reference oligonucleotides that are added to the nucleic acid sample. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few normalization probes are used and they are selected such that they hybridize well (i.e. no secondary structure) and do not match any target-specific probes.

Expression level controls are probes that hybridize specifically with constitutively expressed genes in the biological sample. Expression level controls are designed to control for the overall health and metabolic activity of a cell. Examination of the covariance of an expression level control with the expression level of the target nucleic acid indicates whether measured changes or variations in expression level of a gene is due to changes in transcription rate of that gene or to general variations in health of the cell. Thus, for example, when a cell is in poor health or lacking a critical metabolite the expression levels of both an active target gene and a constitutively expressed gene are expected to decrease. The converse is also true. Thus where the expression levels of both an expression level control and the target gene appear to both decrease or to both increase, the change may be attributed to changes in the metabolic activity of the cell as a whole, not to differential expression of the target gene in question. Conversely, where the expression levels of the target gene and the expression level control do not covary, the variation in the expression level of the target gene is attributed to differences in regulation of that gene and not to overall variations in the metabolic activity of the cell. Typically, expression level control probes have sequences complementary to subsequences of constitutively expressed “housekeeping genes” including, but not limited to the β-actin gene, the transferrin receptor gene, the GAPDH gene, tyrosine 3-monooxygenase/tryptophan 5-monooxygenase activation protein, zeta polypeptide, and/or ubiquitin C and the like.

Mismatch controls may also be provided for the probes to the target genes, for expression level controls or for normalization controls. Mismatch controls are oligonucleotide probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g. stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20 mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross-hybridization to a nucleic acid in the sample other than the target to which the probe is directed. Mismatch probes thus indicate whether hybridization is specific or not. For example, if the target is present the perfect match probes should be consistently brighter than the mismatch probes.

In one embodiment, an array may also include sample preparation/amplification control probes. These are probes that are complementary to subsequences of control genes selected because they do not normally occur in the nucleic acids of the particular biological sample being assayed.

In one embodiment, the oligonucleotide array is hybridized to a sample containing target nucleic acids having subsequences complementary to the oligonucleotide probes and the difference in hybridization intensity between each probe and an appropriate control is determined.

In certain preferred embodiments of the present invention, for example, array analysis may carried out using the GeneChip system of Affymetrix (or other chips that monitor expression of majority of known human RNA transcripts (e.g., HTHGU133plusPM)) following recommended procedures. Hybridization and processing of such GeneChips may be performed using the automated GeneChip Instrument System. Data acquisition, sample normalization, and initial data analysis may be performed with Affymetrix Microarray Suite (MAS) software. In another embodiment, arrays suitable for detection of from 1-5, from 1-10, from 1-15, from 1-20, from 1-30, from 1-40, from 1-50 or from 1-100 IFN inducible genes can be used in the claimed methods.

In one embodiment, the data collected from such array analysis are imported into a computing environment, wherein software and other tools may be used to analyze and interpret such data.

The RNA expression profile of cells from subjects suffering from an autoimmune disorder may then be analyzed, preferably, in pair-wise fashion with a suitable control to identify genes that are significantly overexpressed. In certain preferred embodiments, the quality of data will also be determined.

D. Cytokine/Chemokine Expression

In one embodiment, subjects having increased levels of IFN can be identified by increased levels of expression of certain cytokines or chemokines. For example, the IFN induced chemokines CCL2 (monocyte chemotactic protein 1 [MCP-1]), CCL19 (macrophage inflammatory protein 3β [MIP-3β]), and/or CXCL10 (IFNγ-inducible 10-kd protein [IP-10]) can be detected as markers of increased IFN levels in a subject.

In another embodiment, subjects identified as having an IFN signature can be further screened for levels of expression of cytokines or chemokines using techniques known in the art, e.g., as described in Bauer et al. Arthritis & Rhematology. 2009. 60:3098.

In one embodiment, such detection is done at the protein level, e.g., by measuring the levels of cytokine protein in a biological sample (e.g., in serum or blood) or made by a biological sample (e.g., cells or stimulated cells) from the subject.

Methods for measuring proteins are well known in the art, e.g., ELISA assays or other antibody based assays can be used. In another embodiment, levels of chemokine transcripts can be measured as known in the art or as set forth above for other IFN-inducible genes.

For example, in one embodiment, subjects having increased expression of certain genes induced by IFN and/or certain cytokines are more responsive to treatment with agents that inhibit signaling via LTβR. In another embodiment, subjects having both increased expression of certain genes induced by IFN and certain cytokines are more responsive to treatment with agents that inhibit signaling via LTβR. In one embodiment, the cytokine is a chemokine. For example, expression of one or more chemokines such as CCL9, CCL10, CCL19, CCL21, CCL12, and CCL13 can be measured.

### III. METHODS OF TREATMENT

Given the observation that an increase in IFN or a marker thereof is indicative of an improved therapeutic outcome upon treatment with an agent that reduces or inhibits signaling via LTβR, one of ordinary skill in the art can now select an appropriate treatment regimen for a subject and, optionally, administer an appropriate treatment. Accordingly, in one embodiment, the above-described method for predicting the therapeutic responsiveness of a subject afflicted with an autoimmune disorder to an agent that inhibits signaling via LTβR further comprises selecting a subject for whom such treatment is likely to be effective. In another embodiment, the above-described method for predicting the therapeutic responsiveness of a subject afflicted with an autoimmune disorder to an agent that inhibits signaling via LTβR further comprises selecting a treatment regimen for that subject which employs an agent that inhibits signaling via LTβR. In another aspect, the method further comprises administering the agent that inhibits signaling via LTβR to a subject belonging to the subset selected according to a predictive method described herein to thereby improve the therapeutic outcome for the subject having an autoimmune disorder.

Exemplary autoimmune disorders include: Sjogren's syndrome, scleroderma, lupus, polymyositis/dermatomyositis, cryoglobulinemia, anti-phospholipid antibody syndrome, and psoriatic arthritis), autoimmune gastrointestinal and liver disorders, autoimmune gastritis and pernicious anemia, autoimmune hepatitis, primary biliary cirrhosis, primary sclerosing cholangitis, celiac disease, vasculitis, autoimmune neurological disorders, renal disorders, autoimmune dermatologic disorders, hematologic disorders, atherosclerosis, uveitis, autoimmune hearing diseases, Behcet's disease, Raynaud's syndrome, dermatomtositis, organ transplant, autoimmune endocrine disorders, IBD, and Type I diabetes.

In another embodiment, the autoimmune disorder is selected from the group consisting of: Sjogren's syndrome, lupus, inflammatory myositis, psoriasis, multiple sclerosis, and rheumatoid arthritis (RA).

In still another embodiment, the autoimmune disorder is RA.

A treatment regimen with an agent that inhibits signaling via LTβR typically includes at least one of the following parameters and more typically includes many or all of the following parameters: the type of agent chosen for administration, the dosage, the formulation, the route of administration and/or the frequency of administration.

In one embodiment, the amount of agent that inhibits signaling via LTβR given to the subject can be reduced from that normally given (i.e., the current standard of care) because the subject is more sensitive to treatment with the agent that inhibits signaling via LTβR. In another embodiment, the amount of agent that inhibits signaling via LTβR given to the subject (whether given at the normal dose or at a reduced dose) can be given for a reduced period of time because the selected subject is more sensitive to LTβR blockade. Exemplary protocols for administering agents that inhibit signaling via LTβR are known in the art.

### IV. EXEMPLARY AGENTS THAT INHIBIT SIGNALING VIA LTR

Agents that inhibit signaling via LTβR are known in the art. For example, in one embodiment, a soluble LTβR fusion protein may be used. The soluble LTβR can include the entire extracellular domain of LTβR or a portion thereof which retains the ability to bind to LTβ, e.g., a soluble fragment of LTβR. An exemplary LTβR moiety is the wild-type LTβR sequence or a sequence which differs therefrom by no more than 1, 2, 3, 5, or amino acid residues. The differences can be any difference, e.g., a substitution, deletion or insertion, but is preferably a substitution, e.g., a conservative substitution. Conservative substitutions are usually exchanges of one amino acid for another with similar polarity, steric arrangement, or of the same class (e.g., hydrophobic, acidic or basic). In one embodiment, such molecules may be fused to one or more heterologous protein domains (which domain(s) may increase solubility or lifetime in the blood). Examples of non-LTβR proteins or domains include all or part of the constant region of an antibody, e.g., an Fc domain, transferrin, or albumin, such as human serum albumin (HSA) or bovine serum albumin (BSA).

In a preferred embodiment, the polypeptide of the invention is an Fc fusion protein containing a polypeptide such as an antibody, and preferably an IgG immunoglobulin, e.g., of the subtype IgG1, IgG2, IgG3, or IgG4, and preferably, of the subtype IgG1 or IgG4. In a preferred embodiment, the foregoing polypeptide binds to a ligand of LTβR. Amino acid numberings herein for portions of an Fc region of a polypeptide correspond to the Kabat numbering system as described, e.g., by Kabat et al., in “Sequences of Proteins of Immunological Interest”, U.S. Dept. Health and Human Services, 1983 and 1987. In some embodiments, sequential amino acid numbering, e.g., for sequences presented in the sequence listing, are provided. In one embodiment, a fusion protein of the invention comprises at least a portion of a hinge region, a CH1, a CH2, and a CH3 region of an immunoglobulin.

An example of a wild-type LTβR-Ig fusion protein is set forth below. It should be noted that the terms LTβR-Ig and LTβR-Fc are used interchangeably herein.

In one embodiment, an LTβR-Ig fusion protein comprises a variant LTR extracellular domain and/or a variant Ig portion, e.g., Fc portion of an Ig. In one embodiment of the invention, the LTβR-Ig fusion protein comprises either a LTβR extracellular domain variant, a variant Ig portion, or a combination thereof.

The amino acid and nucleic acid sequences of wild type LTβR are described in the NCBI database as AAH26262 and P36941. The wild type human amino acid sequence of LTβR is set forth below. In a preferred embodiment the soluble LTβR is an LTβR-Fc polypeptide which differs from the sequence of the wild-type sequence by no more than 1, 2, 3, 5, or 10 amino acid residues.

Human LTR Sequence (GenPept ID No. P36941)

The immature or nonprocessed human LTβR sequence, i.e., which contains the signal sequence, is set forth below. Amino acids in italics indicate signal sequence. Amino acids 28-225 are the extracellular region of LTβR.

```
(SEQ ID NO: 1)
    1 MLLPWATSAP GLAWGPLVLG LFGLLAASQP QAVPPYASEN QTCRDQEKEY YEPQHRICCS
  61 RCPPGTYVSA KCSRIRDTVC ATCAENSYNE HWNYLTICQL CRPCDPVMGL EEIAPCTSKR
121 KTQCRCQPGM FCAAWALECT HCELLSDCPP GTEAELKDEV GKGNNHCVPC KAGHFQNTSS
181 PSARCQPHTR CENQGLVEAA PGTAQSDTTC KNPLEPLPPE MSGTMLMLAV LLPLAFFLLL
241 ATVFSCIWKS HPSLCRKLGS LLKRRPQGEG PNPVAGSWEP PKAHPYFPDL VQPLLPISGD
301 VSPVSTGLPA APVLEAGVPQ QQSPLDLTRE PQLEPGEQSQ VAHGTNGIHV TGGSMTITGN
361 IYIYNGPVLG GPPGPGDLPA TPEPPYPIPE EGDPGPPGLS TPHQEDGKAW HLAETEHCGA
421 SNRGPRNQ FITHD
```

The term “wild type LTβR-Ig” as used herein, refers to a fusion protein comprising the extracellular domain of human wild type LTβR, e.g., the mature form of the extracellular domain of the LTβR sequence presented above, and any immunoglobulin sequence known in the art which is not modified, for example, by mutations, deletions, etc.

A particularly preferred soluble LTβR molecule comprises the mature form of the amino acid sequence:

M L L P W A T S A P G L A W G P L V L G L F G L L A A A V P P Y A S E N
Q T C R D Q E K E Y Y E P Q H R I C C S R C P P G T Y V S A K C S R I R
D T V C A T C A E N S Y N E H W N Y L T I C Q L C R P C D P V M G L E E
I A P C T S K R K T Q C R C Q P G M F C A A W A L E C T H C E L L S D C
P P G T E A E L K D E V G K G N N H C V P C K A G H F Q N T S S P S A R
C Q P H T R C E N Q G L V E A A P G T A Q S D T T C K N P L E P L P P E
M S G T M [symbol] D K T H T C P P C P A P E L L G G P S V F L F P P K P K D T
L M I S R T P E V T C V V V D V S H E D P E V K F N W Y V D G V E V H N
A K T K P R E E Q Y N S T Y R V V S V L T V L H Q D W L N G K E Y K C K
V S N K A L P A P I E K T I S K A K G Q P R E P Q V Y T L P P S R D E L
T K N Q V S L T C L V K G F Y P S D I A V E W E S N G Q P E N N Y K T T
P P V L D S D G S F F L Y S K L T V D K S R W Q Q G N V F S C S V M H E
A L H N H Y T Q K S L S L S P G Stop

Amino acids in italics indicate signal sequence which is not present in the mature form of the protein.

A recombinant expression vector containing a soluble LTβR polynucleotide sequence can be introduced into and/or maintained within a cell. Cells expressing a soluble LTβR molecule may be prokaryotic. Alternatively, a soluble LTβR nucleic acid can be introduced into a eukaryotic cell, e.g., a eukaryotic cell that contains the appropriate machinery for post-translational processing of a polypeptide into a mature protein, and/or the appropriate machinery for secreting a polypeptide into the extracellular environment of the cell.

Suitable methods of making LTβR-Ig proteins of the invention are known in the art. For example, an LTβR immunoglobulin fusion protein can be expressed in cell culture (e.g., mammalian cell culture (such as monkey cos cells or Chinese hamster ovary cells) or yeast cell culture) at a reduced temperature, e.g, to produce an increased amount of properly folded fusion protein. Also included within the scope of the invention are host cells expressing LTβR-Ig fusions proteins of the invention, where the host cell comprises a vector comprising a nucleic acid encoding an LTβR-Ig fusion protein. In one embodiment, the host cell is a Chinese Hamster Ovary (CHO) cell. The expressed fusion protein can be purified, e.g., by affinity or conventional chromatography techniques using art recognized methods. Expression of the LTβR-Ig fusion protein may range in scale, for example, may be done at manufacturing scale.

Another type of agent which blocks LTβR signaling is an antibody which binds to LT and blocks the binding of LT to LTβR. In one embodiment, an antibody binds to LTβ. In one embodiment, an antibody binds to surface LTα. In one embodiment, an antibodies bind to LTαβ.

In one embodiment, an antibody binds to LTα3. In another embodiment, an antibody does not bind to LTα3 (or binds to LTα3, but not in such a way as to block TNFα receptor binding). For example, a panel of such antibodies has been developed and the epitopes to which several of these antibodies bind have been mapped (see, e.g., PCT/US2009/069967).

The structure of the variable regions of these antibodies has also been elucidated. The CDRs from this panel of antibodies (e.g., Chothia or Kabat CDRs) can be used to generate binding molecules (e.g., humanized antibodies, modified antibodies, single chain binding molecules) that bind to LT and block LT-induced signaling. Accordingly, in one embodiment, an agent that blocks LTβR signaling is a binding molecule which comprises one or more binding sites (e.g., light and heavy chain CDRs or variable heavy and variable light regions) specific for LT, which block the binding of LT to LTβR.

In another embodiment, an agent that blocks LTβR signaling is a form of the soluble decoy receptor DcR3 (also known as TNFRSF6B) that reduces the binding of LIGHT to LTβR (see, e.g., Wroblewski et al. 2003. Biochem Pharmacol. 65:657).

### V. KITS OF THE INVENTION

The methods described herein may be performed utilizing pre-packaged diagnostic kits comprising at least one reagent for detection of the level of expression of IFN or a marker thereof. For example such kits may comprise a reporter gene, a means of detecting an autoimmune antibody, or a means of detecting at least one IFN-inducible gene, which may be conveniently used, e.g., in clinical settings to identify or select patients exhibiting symptoms or family history of an autoimmune disorder. In addition, a readily available commercial service can be used to analyze samples for markers of the presence of increased levels of IFN.

The kits of the invention may optionally comprise additional components useful for performing the methods of the invention. By way of example, the kits may comprise means for obtaining a biological sample from a subject, a control sample, e.g., a known negative and/or positive control, means for detecting the IFN or a marker of increased expression thereof, and optionally an agent that inhibits LTβR signaling. Such kits may also include instructions for use of the kit.

In one embodiment, the means for detecting the level of expression of at least one IFN-inducible gene comprises an array or microarray. In one embodiment, this array includes at least one, and may include more than one, nucleic acid probe, the sequence(s) of which is designed such that the level of expression of at least one IFN-inducible gene may be measured.

The kit can also include, for example, reagents for use in an assay for evaluating gene expression (e.g., at either the mRNA or protein level).

The means for isolating a biological sample from a subject can comprise one or more reagents that can be used to obtain a tissue from a subject, such as means for obtaining a biopsy.

In another embodiment, the kit can further comprise an agent that inhibits LTβR signaling for treating an autoimmune disorder in the subject.

Preferably, the kit is designed for use with a human subject.

The contents of all references, pending patent applications and published patents, cited throughout this application are hereby expressly incorporated by reference. Each reference disclosed herein is incorporated by reference herein in its entirety. Any patent application to which this application claims priority is also incorporated by reference herein in its entirety. The contents of the attached appendix are specifically incorporated herein by this reference.

This invention is further illustrated by the following examples which should not be construed as limiting.

### EXAMPLES

The following Materials and Methods were used in the Examples:

### Methods

### Patients:

The RA203 trial enlisted 115 patients in the double-blinded placebo-controlled clinical trial with 77 treated patients and 38 placebos. All patients were TNF-IR, had no adequate response to previous TNF-blocking therapy and were no longer receiving TNF-blocking treatment. Treated patients received single dose of soluble LTβR (in the form of an LTβR Ig fusion protein) subcutaneously (SC) 200-mg bi-weekly for 14 weeks. All patients consented to participate in the trail and subsequent molecular analysis of the blood samples.

The RA202 double-blinded placebo controlled trial, enlisted RA patients who were methotrexate inadequate responders (MTX-IR). 391 patients (79 placebos and 312 patients treated with increasing doses of soluble LTβR) were enlisted in this trial. There were 78 patients treated with 5 mg of soluble LTβR every other week (eow), 39 with 70 mg monthly, 78 with 70 mg eow, 39 with 200 mg monthly and 78 with 200 mg eow.

Blood Measurements

Lymphocyte counts were measured using conventional clinical cell counters as part of the clinical study. Plasma or serum was collected for chemokine quantitation. In some case, single chemokines were determined using commercial ELISA kits. All samples were also measured used a custom developed Luminex multiplex assay (Rules Based Medicine).

### Collection of Blood Samples for RNA Profile:

Patient whole blood was collected pre-treatment (week 0), after 6 and after 14 weeks of treatment for RA203 and for week 0 and 14 weeks of treatment for RA202 samples. The RNA was extracted from whole blood collected in PAXgene tubes, processed and hybridized to Affymetrix microarrays. The RNA was profiled using the high-throughput Affymetrix chips HTHGU133plusPM that monitor expression of majority of known human RNA transcripts (Allaire N E, Rieder L E, Bienkowska J, & Carulli J P (2008) Genomics 92(5):359-365).

RNA profiles from each of the two trials were normalized separately using the GCRMA method implemented in BioConductor. Next, QC analysis was performed using standard protocols available from BioConductor. Samples with high variation in normalized un-scaled standard error (NUSE) and relative log error (RLE) were removed. Samples with high RNA degradation rates (>4) were also excluded from further analysis. Paired sample analysis (see below) was restricted to those pairs with similar RNA degradation rates (RNA degradation slope differences <1). After QC of clinical and molecular data for the RA203 trial, 27 and 44 paired samples for placebos and treated patients for weekl4-week0 differences remained. For week6-week0 differences, 30 and 44 paired samples were used for placebos and treated patients in RA203 trial. For RA202 samples, after QC data for week 0 and week 14 for 47 placebos, and 5 patient cohorts treated with increasing doses of soluble LTβR: 46 patients treated with 5 mg of soluble LTβR every-other-week (eow), 44 patients treated with 70 mg soluble LTβR monthly, 26 patients treated with 70 mg eow, 25 patients treated with 200 mg soluble LTβR monthly and 46 patients treated with 200 mg soluble LTβR eow remained.

### Paired-Sample Analysis:

The GCRMA gene expression values (log 2-transformed) were used in this analysis. Differences in gene expression between week 14 and week 0, week 6 and week 0, were calculated in treated patients using the paired sample approach. First, for each patient a gene expression difference was calculated. Second, for the group of treated patients, defined by the treatment dose, whether the observed differences are significantly different than 0 was determined. For this analysis, differences of at least 1.5 fold and having a p value <0.05, were considered significantly regulated. Once genes significantly regulated in any group of treated patients were identified, a hierarchical clustering using the differences calculated for placebos was used and subgroups of patients similar to placebos and those that differ from placebos were identified.

### Differential Gene Expression Between Groups of Patients:

To identify gene expression differences between any two groups of samples the linear modeling approach implemented in limma package from BioConductor was used. To determined significant differences of expressed genes, fold differences greater than 1.5 fold and having a p value (Bayesian prior corrected as implemented in limma) less than 0.05 were identified.

### Linear Models of Soluble LT/3R Effect on IFN Signature:

To assess whether the soluble LTβR treatment had a significant effect on attenuation of the IFN signature as compared to placebos, in one embodiment, a linear modeling approach was used. The observed changes in the interferon signature (AIFN) were used as a function of the baseline IFN score: IFN0, treatment: RX (placebo vs. soluble LTβR treatment) and combination of baseline IFN0 score and treatment RX:IFN0. The linear model_represents this hypothesis. Standard general linear modeling ANOVA analysis was used to assess the significance of the hypothesis that both baseline IFN0 signature and soluble LTβR treatment are significantly correlated with attenuation of the IFN signature.

### Example 1

### Soluble LTR Treatment Up-Regulates B-Cell Signature Genes in a Sub-Group of RA Patients

Samples from subjects treated with LTβR fusion protein have been used to investigate whether there was an observable change in molecular profiles of treated patients after the 6 and/or 14 weeks treatment. First, mRNA expression was profiled in the whole blood collected pre- and after 6 and 14 weeks of treatment in 114 TNF non-responder patients, 37 placebo and 77 treated with single bi-weekly dose of LTβR fusion protein at 200 mg subcutaneously (SC). Using paired samples analysis, 124 genes that are differentially regulated by LTβR were identified after 6 or 14 weeks of treatment. The majority of 78 genes that were up-regulated by LTβR fusion protein represent B-cells expressed genes, while among the 46 down-regulated genes were several interferon induced genes (Hilpert J, et al. (2008) J Neuroimmunol 199(1-2):115-125). Among the 78 up-regulated genes 43 were up-regulated at both time points with additional 27 up-regulated after 14 weeks treatment. Among the 46 down-regulated genes 13 were up regulated at both time points with additional 34 down-regulated after 14 weeks of treatment.

Similarly, differentially regulated genes in the blood collected from Methotrexate-non-responder patients in RA202 were examined. In this trial patients were treated with 5 increasing doses of LTβR fusion protein or placebo. LTβR fusion protein significantly changed expression of 84 genes. Consistent with the observation from the TNF non-responder trial the up-regulated genes reflected up-regulation of the B-Cell expressed genes. However, among the down regulated genes none is known to be IFN-regulated.

Using the unsupervised hierarchical clustering of 124 genes expression differences we identified two subgroups of LTβR fusion protein treated patients in the RA203 trial. Clustering revealed one subgroup of soluble LTβR treated patients shows gene expression differences after treatment similar to those observed in placebos (no significant gene expression regulation) and a second group that is characterized by up-regulations of the B-Cell genes and down-regulation of several IFN signature genes. This patients clustering was most pronounced in the week14-week 0 differences but was already evident after 6 weeks of treatment.

A similar unsupervised clustering approach was applied using 84 genes differentially expressed in the RA202 trial. The clustering revealed sub-grouping of patients into two classes similar to the RA203 trial observation. One subset of patients responded to soluble LTβR therapy with up-regulation of B-Cell genes and the second subset of patient exhibiting changes in gene expression similar to those observed in placebos. Interestingly, with the decreasing doses of soluble LTβR the percentages of patients responding with B-Cell up-regulation decreases from 80%, to 60% to 45% for the top three doses of soluble LTβR, 200 mg eow, 200 mg monthly and 70 mg eow.

### Example 2

### Patients Who Respond to Soluble LTβR Treatment have Up-Regulated IFN-Signature

Following the observation of two distinct molecular response groups in treated TNF-non-responder patients, these two groups were named BCellH and BCellL patients, reflecting the strong up-regulation of the B-Cell genes in the first group and lack of such regulation in the second. Comparison of the gene expression differences at baseline (week0) between BCellH and BCellL groups identified 15 genes as significantly different: OAS3, HERC5, OAS1, TIMM10, RSDA2, IFI44L, IFI44, IFI6, IFIT3, ISG15, MX1, DOX58, UBE2L6, BATF2, LIPA (see Table 1). These genes have been reported in literature as being regulated by IFN-a treatment (Hilpert et al) and are also known as IFN signature genes shown by others to be often up-regulated in autoimmune disorders (RA, SLE, Sjogren Syndrome, MS) when compared to healthy controls (Hilpert J, et al. (2008) J Neuroimmunol 199(1-2):115-125; Yao Y, et al. (2009). Human Genomics and Proteomics:1-16).

Similarly, two types of molecular response were observed in the MTX-IR patients treated with the decreasing doses of soluble LTβR. Owing to dose effects seen as the diseasing percentage of responders, the BCellH and BCellL patients for each dose were identified separately. As expected most of genes differentially expressed at baseline are found for the highest dose of soluble LTβR treatment. Among the 16 genes selected in RA203 trail analysis, eight are differentially expressed at baseline between BCellH and BCellL MTX-IR patients. Additionally, several other IFN-regulated genes (Hilpert J, et al. (2008) Biological response genes after single dose administration of interferon beta-1b to healthy male volunteers. J Neuroimmunol 199(1-2):115-125) are differentially expressed, likely indicating differences between these two patient populations. Interesting genes expressed by NK-cells KR3DL2 and KIR3DL3 are also differentially expressed at baseline between the MTX-IR responder and non-responder group. Owing to the higher variability of the MTX-IR data, the significance correction for multiple hypothesis testing was not applied.

### Example 3

### Soluble LTβR Treatment Attenuates IFN Gene Signatures

After identification of soluble LTβR responsive patients by unsupervised clustering, differential gene regulation was reanalyzed in soluble LTβR in the BCellL and BCellH TNF-non responder patient groups. Using the linear modeling approach, genes significantly regulated in BCellH patients after 14 weeks of treatment were identified. IFN-signature genes are down-regulated by soluble LTβR 2-5 fold only in the BCellH patient group. By definition this group is characterized by significant up-regulation of B-Cell genes by soluble LTβR treatment, while just a few B-cell genes are up-regulated in BCellL group with fold changes less than 2. All 16 IFN-signature transcripts differentially expressed between these two groups of patients at baseline are significantly down-regulated by soluble LTR in the BCellH group only, with down-regulation already significant after 6 weeks of treatment.

It has also been confirmed that soluble LTβR treatment leads to significant attenuation of the IFN signature in this patient cohort from the RA202 trial. Similar to the RA203 trial, sub-groups of patients treated with soluble LTβR respond with up-regulation of the B-Cell expressed genes. With dose escalation a greater number of B-Cell expressed genes are up-regulated by treatment and increasing fractions of patients treated with escalating doses respond with up-regulation of these genes. In the top highest soluble LTβR doses significant up-regulation of at least some of B-Cell genes was observed with many of the significantly regulated genes the same across the dose cohorts.

Using the 16—gene signature set identified in the RA203 trial it has been found that the elevated pre-treatment IFN signature is indicative of the response to soluble LTβR treatment. The geometric mean expression of the 16 genes pre- and 14-weeks post-treatment has been calculated to represent a single IFN score for both TNF-IR and MTX-IR patients. The MTX-non responder patients were grouped as on treatment group for four top soluble LTβR doses and evaluated 5 mg eow dose separately from placebo. A linear modeling approach was used to assess the significance of the soluble LTβR treatment on the IFN score. Table 2 summaries the ANOVA analysis of the soluble LTβR effect on the ΔIFN, the difference in the IFN score between 14-weeks treatment and baseline. Both baseline IFN signature (IFN0) and treatment significantly correlate with the IFN attenuation. The P value for the soluble LTβR treatment (soluble LTβR:IFN0) significantly affecting IFN signature is 10⁻⁴ and 3*10⁻² for the methotrexate non responder and TNF non responder trail. The effect of soluble LTβR treatment is more significant in the TNF-non responder patient cohort.

### Example 4

### Soluble LTβR Normalizes Elevated Cytokines Levels Correlated with Elevated IFN Signature

Several cytokines correlated with the IFN signature are regulated by soluble LTβR treatment in TNF-IR patients. Using the baseline IFN+/− signature classification of patients we have analyzed the regulation of CXCL9, CCL19, CCL21 and CXCL13 in serum of TNF-IR patients treated with soluble LTβR 200 mg eow or Placebo. The serum levels of all cytokines correlate with the IFN signature and soluble LTβR significantly down regulates their expression. For all 4 of those the post-treatment levels are similar to those observed in healthy controls.

### Example 5

### Soluble LTβR Decreases Swollen Joint Counts in Patients with Elevated IFN Signature

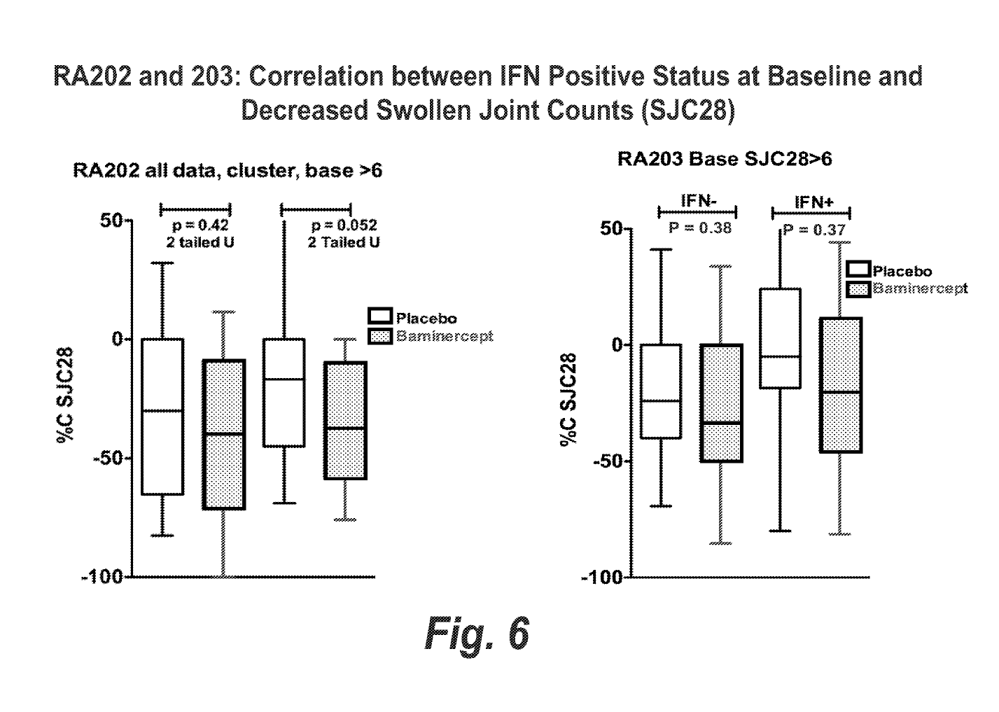

Using the patient groups defined by the baseline IFN+/− signature we have analyzed the effects of soluble LTβR on the percentage changes in Swollen Joint Counts 28. Soluble LTβR has no effect on % change SJC28 in IFN-patients when compared to placebos with the similar IFN-signature status. In the group of IFN+ patients there is a clear trend towards the significant reduction in % change SCJ28 with the p.value 0.07 between the Placebo and treatment. Lack of significance is likely due to small number of IFN+ patients represented in this group of patients. The effects of soluble LTβR on % change SJC28 in the MTX-IR patients with IFN+/− baseline signatures have been examined. To overcome the limitations of small number of patients treated with different doses of soluble LTβR in this trial patients treated with 4 highest doses have been grouped. In the group of patients with IFN-signature soluble LTβR has no significant effect on % SJC when compared to placebos with similar signature (see FIG. 6B). Notably the comparison of soluble LTβR effects in the IFN+ group shows a significant effect of soluble LTβR treatment when compared to placebos, with the p.value of 0.005. This observation indicates that besides significant effects on molecular markers such as elevated IFN signature genes, IFN regulated cytokines and lymphopenia, soluble LTβR alleviates the clinical manifestations of the RA in patients with up-regulated IFN signature.

TABLE 1
Genes differentially expressed at baseline between patients responding vs. non-
responding to soluble LTβR treatment. Column 3 shows fold differences between BCellH
and BCellL group. Columns 3 and 4 show fold change within BCellH group only after 6
and 14 weeks of treatment and columns 5 and 6 show the p.value for the week 6 and 14
differences when compared to variation observed in placebos.

| | | Fold Changes | | | Pvalue vs. Placebos | |
|---|---|---|---|---|---|---|
| Probeset | SYMBOL | W0_BCellH_BCellL | W6_BCellH | W14_BCellH | W6_BCellH | W14_BCellH |
| 242625_at | RSAD2 | 4.87 | 4.74 | 5.30 | 9.59E−04 | 5.51E−04 |
| 204439_at | IFI44L | 4.72 | 4.12 | 5.31 | 3.76E−03 | 1.15E−03 |
| 213797_at | RSAD2 | 4.43 | 4.11 | 4.16 | 1.91E−03 | 1.81E−03 |
| 236156_at | LIPA | 3.28 | 3.67 | 3.76 | 4.80E−03 | 4.28E−03 |
| 205483_s_at | ISG15 | 3.26 | 3.44 | 3.02 | 1.69E−03 | 3.71E−03 |
| 218400_at | OAS3 | 3.24 | 3.04 | 3.71 | 1.39E−03 | 3.72E−04 |
| 228439_at | BATF2 | 3.22 | 2.59 | 3.48 | 2.03E−02 | 4.43E−03 |
| 204747_at | IFIT3 | 3.11 | 3.21 | 3.16 | 3.52E−03 | 3.82E−03 |
| 204415_at | IFI6 | 2.99 | 2.71 | 2.97 | 6.41E−03 | 3.70E−03 |
| 219863_at | HERC5 | 2.91 | 2.80 | 3.03 | 9.09E−04 | 5.03E−04 |
| 214453_s_at | IFI44 | 2.84 | 2.77 | 3.27 | 4.56E−04 | 1.19E−04 |
| 205552_s_at | OAS1 | 2.60 | 2.52 | 2.41 | 1.87E−03 | 2.66E−03 |
| 202086_at | MX1 | 2.60 | 2.21 | 2.53 | 4.63E−03 | 1.55E−03 |
| 1555764_s_at | TIMM10 | 1.84 | 1.80 | 1.79 | 4.29E−03 | 4.52E−03 |
| 218943_s_at | DDX58 | 1.72 | 1.77 | 1.85 | 1.38E−03 | 8.16E−04 |
| 201649_at | UBE2L6 | 1.52 | 1.48 | 1.54 | 2.12E−02 | 1.32E−02 |

TABLE 2
The results of ANOVA analysis of the linear modeling of IFN
signature down-regulation by soluble LTβR treatment (RX row).

| | RA202 | | RA203 | |
|---|---|---|---|---|
| | Df | p.value | Df | p.value |
| W0 | 1 | <2e−16 | 1 | 2.11E−11 |
| RX | 2 | 2.77E−01 | 1 | 1.97E−03 |
| W0:RX | 2 | 2.98E−02 | 1 | 1.01E−04 |
| Residuals | 228 | | 47 | |

What is claimed is:

1. A method for predicting the responsiveness of a subject having an autoimmune disorder to a treatment with an agent that inhibits LTβR-mediated signaling, the method comprising,
a) contacting a biological sample from the subject with a reagent allowing detection of increased levels of IFN or a marker thereof;
b) detecting the level of IFN or a marker thereof, wherein the presence of an increased level of IFN or a marker thereof as compared to an appropriate control indicates that the patient will likely respond to therapy with an agent that inhibits LTβR-mediated signaling.

2. The method of claim 1, wherein the biological sample from the subject is contacted with a reagent to obtain a detectable composition allowing detection of the level of IFN or a marker thereof.

3. The method of claim 1, wherein increased levels of IFN are detected.

4. The method of claim 1, wherein increased levels of one or more genes induced by IFN are detected.

5. The method of claim 1, wherein increased levels of one or more autoantibodies indicating the increased expression of IFN are detected.

6. The method of claim 1, wherein increased levels of one or more cytokines indicating the increased expression of IFN are detected.

7. The method of claim 1, further comprising treating the subject with an agent that inhibits LTβR-mediated signaling.

8. The method of claim 1, wherein the biological sample is a plasma sample.

9. The method of claim 1, wherein the biological sample is a blood sample.

10. The method of claim 1, wherein the biological sample comprises cells.

11. The method of claim 10, wherein the biological sample is manipulated prior to the step of contacting.

12. The method of claim 11, wherein the biological sample comprises extracted nucleic acid molecules.

13. The method of claim 1, wherein the reagent comprises a nucleic acid molecule which hybridizes to a nucleic acid molecule derived from the transcript of at least one gene induced by IFN.

14. The method of claim 13, wherein the at least one gene is selected from the group consisting of: OAS3, HERC5, OAS1, TIMM10, RSDA2, IFI44L, IFI44, IFI6, IFIT3, ISG15, MXI, DOX58, UBE2L6, BATF2, and LIPA.

15. The method of claim 1, wherein the level of expression is determined by measuring transcription of a plurality of genes induced by IFN.

16. The method of claim 15, wherein the level of transcription of at least 5 genes induced by IFN is detected.

17. The method of claim 15, wherein the level of transcription of at least 10 genes induced by IFN is detected.

18. The method of claim 15, wherein the level of transcription of the OAS3, HERC5, OAS1, TIMM10, RSDA2, IFI44L, IFI44, IFI6, IFIT3, ISG15, MXI, DOX58, UBE2L6, BATF2, and LIPA genes are detected.

19. The method of claim 1, wherein the level of expression of at least one chemokine in the subject is measured.

20. The method of claim 13, further comprising measuring the level of expression of at least one chemokine in the subject.

21. The method of claim 19 or 20, wherein the at least one chemokine is selected from the group consisting of CXCL10, CCL19, and CCL2.

22. The method of claim 1, wherein the reagent comprises a reporter cell.

23. The method of claim 1, wherein the reagent is capable of detecting the presence of an autoantibody.

24. The method of claim 1, wherein the autoimmune disorder is selected from the group consisting of: rheumatoid arthritis, Sjogren's syndrome, scleroderma, lupus, polymyositis/dermatomyositis, cryoglobulinemia, anti-phospholipid antibody syndrome, and psoriatic arthritis), autoimmune gastrointestinal and liver disorders, autoimmune gastritis and pernicious anemia, autoimmune hepatitis, primary biliary cirrhosis, primary sclerosing cholangitis, celiac disease, vasculitis, autoimmune neurological disorders, renal disorders, autoimmune dermatologic disorders, hematologic disorders, atherosclerosis, uveitis, autoimmune hearing diseases, Behcet's disease, Raynaud's syndrome, dermatomtositis, organ transplant, autoimmune endocrine disorders, IBD, and Type I diabetes.

25. The method of claim 1, wherein the autoimmune disorder is selected from the group consisting of: RA, Sjogren's syndrome, lupus, inflammatory myositis, psoriasis, multiple sclerosis, and rheumatoid arthritis.

26. A method for treating a subject having an autoimmune disorder with an agent that blocks LTβR-mediated signaling, the method comprising,
a) contacting a biological sample from the subject with a reagent allowing detection of increased levels of IFN or a marker thereof;
b) detecting the level of IFN or a marker thereof, wherein the presence of an increased level of IFN or a marker thereof as compared to an appropriate control indicates that the patient will likely respond to therapy with an agent that inhibits LTβR-mediated signaling;
c) selecting a patient having increased levels of IFN or a marker thereof for treatment with an agent that inhibits LW-mediated signaling.

27. The method of claim 26, further comprising administering an agent that inhibits LTβR-mediated signaling to the subject.

28. The method of claim 27, wherein agent that blocks LTβR-mediated signaling is selected from the group consisting of: a soluble form of an LTβ receptor, and antibody which binds to the LTβ receptor, and an antibody which binds to cell surface LTβ, an antibody that binds to LTα and inhibits the binding of LTβR to cell surface LTα.β, a form of the soluble decoy receptor DcR3 that reduces the binding of LIGHT to LTβR.

29. The method of claim 26, wherein the biological sample from the subject is contacted with a reagent to obtain a detectable composition allowing detection of the level of IFN or a marker thereof.

30. The method of claim 26, wherein increased levels of IFN are detected.

31. The method of claim 26, wherein increased levels of one or more genes induced by IFN are detected.

32. The method of claim 26, wherein increased levels of one or more autoantibodies are detected.

33. The method of claim 26, wherein increased levels of one or more cytokines are detected.

34. The method of claim 26, further comprising treating the subject with an agent that inhibits LTβR-mediated signaling.

35. The method of claim 26, wherein the biological sample is a plasma sample.

36. The method of claim 26, wherein the biological sample is a blood sample.

37. The method of claim 26, wherein the biological sample comprises cells.

38. The method of claim 37, wherein the biological sample is manipulated prior to the step of contacting.

39. The method of claim 38, wherein the biological sample comprises extracted nucleic acid molecules.

40. The method of claim 26, wherein the reagent comprises a nucleic acid molecule which hybridizes to a nucleic acid molecule derived from the transcript of at least one gene induced by IFN.

41. The method of claim 40, wherein the at least one gene is selected from the group consisting of: OAS3, HERC5, OAS1, TIMM10, RSDA2, IFI44L, IFI44, IFI6, IFIT3, ISG15, MXI, DOX58, UBE2L6, BATF2, and LIPA.

42. The method of claim 26, wherein the level of expression is determined by measuring transcription of a plurality of genes induced by IFN.

43. The method of claim 42, wherein the level of transcription of at least 5 genes induced by IFN is detected.

44. The method of claim 42, wherein the level of transcription of at least 10 genes induced by IFN is detected.

45. The method of claim 42, wherein the level of transcription of the OAS3, HERC5, OAS1, TIMM10, RSDA2, IFI44L, IFI44, IFI6, IFIT3, ISG15, MXI, DOX58, UBE2L6, BATF2, and LIPA genes are detected.

46. The method of claim 26, wherein the level of expression of at least one chemokine in the subject is measured.

47. The method of claim 40, further comprising measuring the level of expression of at least one chemokine in the subject.

48. The method of claim 46 or 47, wherein the at least one chemokine is selected from the group consisting of CXCL10, CCL19, and CCL2.

49. The method of claim 26, wherein the reagent comprises a reporter cell.

50. The method of claim 26, wherein the reagent is capable of detecting the presence of an autoantibody.

51. The method of claim 26, wherein the autoimmune disorder is selected from the group consisting of: rheumatoid arthritis, Sjogren's syndrome, scleroderma, lupus, polymyositis/dermatomyositis, cryoglobulinemia, anti-phospholipid antibody syndrome, and psoriatic arthritis), autoimmune gastrointestinal and liver disorders, autoimmune gastritis and pernicious anemia, autoimmune hepatitis, primary biliary cirrhosis, primary sclerosing cholangitis, celiac disease, vasculitis, autoimmune neurological disorders, renal disorders, autoimmune dermatologic disorders, hematologic disorders, atherosclerosis, uveitis, autoimmune hearing diseases, Behcet's disease, Raynaud's syndrome, dermatomtositis, organ transplant, autoimmune endocrine disorders, IBD, and Type I diabetes.

52. The method of claim 26, wherein the autoimmune disorder is selected from the group consisting of: RA, Sjogren's syndrome, lupus, inflammatory myositis, psoriasis, multiple sclerosis, and rheumatoid arthritis.

53. A method for evaluating the response of a subject having an autoimmune disorder to treatment with an agent that blocks LTβR-mediated signaling, the method comprising,
a) contacting a biological sample from the subject with a reagent allowing detection of increased levels of IFN or a marker thereof;
b) detecting the level of IFN or a marker thereof, wherein the presence of an increased level of IFN or a marker thereof as compared to an appropriate control indicates that the patient will likely respond to therapy with an agent that blocks LTβR-mediated signaling;
c) administering an agent that blocks LTβR-mediated signaling to the subject;
d) contacting a second biological sample from the subject taken after step c) with a reagent allowing detection of increased levels of IFN or a marker thereof; wherein the presence of a decreased level of IFN or a marker thereof as compared to the level obtained in step a) indicates that the patient will likely respond to therapy with an agent that blocks LTβR-mediated signaling.

54. A method selecting a treatment regimen for a subject having an autoimmune disorder to a treatment with an agent that inhibits LTβR-mediated signaling, the method comprising,
a. contacting a biological sample from the subject with a reagent allowing detection of increased levels of IFN or a marker thereof;
b. detecting the level of IFN or a marker thereof, wherein the presence of an increased level of IFN or a marker of the expression thereof as compared to an appropriate control indicates that the patient will likely respond to therapy with an agent that inhibits LTβR-mediated signaling;
c. selecting a treatment regimen for the subject employing an agent that blocks LTβR-mediated signaling.

55. Use of an agent that blocks LTβR-mediated signaling for treatment of an autoimmune disorder in a subject, wherein the subject exhibits increased levels of IFN or a marker thereof.

56. Use of an agent that blocks LTβR-mediated signaling in the manufacture of a medicament for treatment of an autoimmune disorder in a subject, wherein the subject exhibits increased levels of IFN or a marker thereof.

57. A kit for predicting therapeutic responsiveness of a subject afflicted with an autoimmune disorder to an agent that blocks LTβR-mediated signaling, the kit comprising a means for determining the level the level of IFN or a marker thereof in a biological sample from the subject.

58. The kit of claim 56, wherein the kit comprises a means of detecting at least one gene regulated by IFN in a biological sample obtained from said subject and instructions for using the kit to predict therapeutic responsiveness of the subject having an autoimmune disorder to an agent that blocks LTβR-mediated signaling.

59. The kit of claim 57, wherein the kit comprises a means for determining the level of a plurality of genes regulated by IFN in the sample.
